(19) United States
(12) Patent Application Publication
(10) Pub. No.: US 2016/0032387 A1
(43) Pub. Date: Feb. 4, 2016

(54) METHODS AND SYSTEMS FOR EVALUATING GENES

(71) Applicant: SYROS PHARMACEUTICALS, INC., Watertown, MA (US)

(72) Inventors: Christian C. Fritz, Natick, MA (US); David A. Orlando, Somerville, MA (US); Matthew G. Guenther, Boston, MA (US); Jakob Loven, Belmont, MA (US); Peter B. Rahl, Natick, MA (US); Charles Y. Lin, Boston, MA (US)

(21) Appl. No.: 14/777,083

(22) PCT Filed: Mar. 14, 2014
(86) PCT No.: PCT/US14/27682

§ 371 Date: Sep. 15, 2015

Related U.S. Application Data
(60) Provisional application No. 61/793,068, filed on Mar. 15, 2013.

(51) Int. Cl. C12Q 1/68; G06F 19/20

(52) U.S. Cl. CPC C12Q 1/6883; C12Q 1/6886; G06F 19/20; C12Q 2600/158; C12Q 2600/112

(57) ABSTRACT

Aspects and embodiments disclosed herein are directed to providing methods and systems for evaluating target genes and/or associated super enhancers and or super-enhance components. In some embodiments, the method includes selecting a gene associated with a super-enhancer, to provide a selected gene, evaluating the selected gene for contribution to a cell state, e.g., a state characterized by a disease or disorder (a disease/disorder state), and responsive to the evaluation, classifying the selected gene and/or associated super-enhancer.

### CLAIM OF PRIORITY

This application claims priority to U.S. Ser. No. 61/793,068, filed Mar. 15, 2013, the content of which is incorporated herein by reference.

### BACKGROUND

1. Field of the Disclosure

Aspects of the present disclosure relate to evaluating genes.

2. Description of Background

A variety of mechanisms to control gene expression are critical in developing and maintaining specific cell types and in health and disease. A more detailed understanding of the elements of gene control will allow advances in the study and treatment of many disorders. By way of example, unwanted gene expression plays a key role in many cancers. An understanding of the elements leading to such unwanted is critical to developing and implementing treatments.

### SUMMARY

Enhancers, which are short cis-acting control elements, exist in two different classes. These two classes play different roles in gene expression. E.g., in embryonic stem cells (ES cells) and in cancer cells, enhancers occur in two different classes: normal enhancers (which have on average low occupancy of Med1 co-activator) and super-enhancers (which have a comparatively high occupancy of Med1 co-activator). In ES cells for example, out of about 6500 enhancers only 211 are super-enhancers (SEs) but out of all the Med1 protein bound to the 6500 enhancers, 40% of the protein is associated with the 211 super-enhancers.

SEs are clusters of enhancers of exceptional size and which are associated with exceptional amounts of transcriptional components. SEs drive high level of expression of associated genes. SEs are found in all mammalian cells and are cell type specific. For example, ES cells have about 200 SEs, pro B cells have 340 SEs, but the overlap (common SEs) is only 15. When SEs are analyzed in different cell types, the SEs map to genes, the top gene ontology (GO) categories of which are found to closely correlate with the cell type analyzed. For example, in ES cells SE regulated genes are associated with embryonic morphogenesis and stem cell development. In contrast, in myotubes, the SEs are preferentially associated with genes involved in muscle tissue and organ development.

In summary, super-enhancers are clusters of enhancers formed by binding high levels of transcription factors and co-activators (such as Med1). SEs drive high levels of transcription but are not associated with highly transcribed housekeeping genes. Mammalian cells (including cancer cells) contain SEs and since SEs preferentially drive cell identity genes, SE analysis provides a new approach to model core regulatory circuits in mammalian cells.

Aspects and embodiments disclosed herein are directed to providing methods and systems for evaluating target genes and/or associated super enhancers and or super-enhance components. In some embodiments, the method includes selecting a gene associated with a super-enhancer, to provide a selected gene, evaluating the selected gene for contribution to a cell state, e.g., a state characterized by a disease or disorder (a disease/disorder state), and responsive to the evaluation, classifying the selected gene and/or associated super-enhancer.

In some embodiments, the method includes classifying the selected gene as a candidate target gene, e.g., for the treatment or diagnosis of a subject having a disease/disorder state, e.g., cancer, e.g., a solid tumor, e.g., lung, ovarian, breast, prostate, skin colon, pancreatic, liver, or kidney cancer, or a heme-cancer, AML, inflammation, or a metabolic disease, e.g., diabetes, obesity, and metabolic syndrome.

In some embodiments, the selected gene is selected responsive to, or at least in part because of, its association with the super-enhancer.

In some embodiments, the method further includes providing a pool of genes associated with super-enhancers. In some embodiments, the selected gene is selected from the pool.

In some embodiments, the cell state is selected from a state of health or disease or disorder, e.g., cancer, e.g., a solid tumor, e.g., lung, ovarian, breast, prostate, skin colon, pancreatic, liver, or kidney cancer, or heme-cancers, e.g., AML, inflammation, or a metabolic disease, e.g., diabetes, obesity, and metabolic syndrome, and/or a state of differentiation, e.g., a less than fully committed cell, e.g., a stem cell, a cancer stem cell, or a precursor cell.

In some embodiments, evaluating includes acquiring knowledge of the selected gene relevant to the cell state.

In some embodiments, evaluating includes acquiring knowledge of the association, correlation, or role of the selected gene to the cell state, e.g., cancer. In some embodiments, the knowledge comprises knowledge of the effect of one or more of the following factors on the cell state, e.g., cancer, or on a parameter associated with the cell state, e.g., proliferation, invasiveness, epithelial to mesenchymal transition, likelihood to metastasize, or cell death:

a) the effect of modulation, e.g., down modulation or up modulation, of the level of gene expression, or of a gene product on the cell state;

b) the effect of gene targeting, e.g., by anti-sense or RNAi, of the selected gene;

c) the effect or results of a synthetic lethal screen;

d) the effect of a genetic event involving the selected gene, e.g., a copy number alteration, e.g., amplification or deletion, a rearrangement, e.g., a translocation, deletion, insertion, or point mutations, or other mutation;

e) expression or hybrid capture sequencing data, e.g., from a source such as the Cancer Cell Line Encyclopedia;

f) the effect of overexpressing a dominant negative mutant of the selected gene; or

g) the effect of reducing the copy number of the gene by genome editing or introducing a analog sensitive mutation into the gene by genome editing. In some embodiments, the method includes acquiring the knowledge from a database, e.g., a public database.

In some embodiments, the method includes determining if a super-enhancer is associated with the target gene in samples from one or a plurality of subjects, e.g., from a plurality of cancer samples of the same or different types.

In some embodiments, the method further includes confirming, e.g., by performing experiments, one of the effects listed above.

In some embodiments, the method further includes evaluating the effect of knockdown by RNAi or overexpression of a dominant negative mutant of the target gene, on the cell state, e.g., in a cell-free, cellular, or animal model.

Aspects and embodiments are also directed to a method of selecting a candidate super-enhancer component. In some embodiments the method includes selecting an enhancer component, e.g., a protein, e.g., a nuclear protein, that is associated with an enhancer, e.g., a transcription factor or chromatin modulator, to provide a selected enhancer component, e.g., a protein that binds to a chromatin domain provided in Table 1 or variant thereof, evaluating the selected enhancer component for contribution to a cell state, e.g., a state characterized by a disease or disorder (a disease/disorder state), and responsive to the evaluation, classifying the selected enhancer component as a candidate super-enhancer component. Table 1 identifies enhancer components that can be used in this analysis. In an embodiment, the component of Table 1 is not one of KDM1, TCF3, MBD3, SMC1A, SMC1B, EP300, CHD4, HDAC1, NR5A2, MED1, SMARCA4, MYOD1, KLF4, BRD4, PRDM14, FOXO1, NIPBL, SMAD3, CHD7, SOX2, MED12, HDAC2, POU5F1, SIRT1, DOT1L, or CEBPA.

In some embodiments, the method further includes providing a pool of enhancer components, e.g., an enhancer component that contains a chromatin domain provided in Table 1 or variant thereof. In an embodiment, the component of Table 1 is not one of KDM1, TCF3, MBD3, SMC1A, SMC1B, EP300, CHD4, HDAC1, NR5A2, MED1, SMARCA4, MYOD1, KLF4, BRD4, PRDM14, FOXO1, NIPBL, SMAD3, CHD7, SOX2, MED12, HDAC2, POU5F1, SIRT1, DOT1L, or CEBPA.

In some embodiments, the selected enhancer component is selected from the pool.

In some embodiments, the method includes determining the identity of or finding an enhancer component by searching a database, e.g., a public database.

In some embodiments, the method includes classifying the super-enhancer component as a candidate therapeutic or diagnostic target, e.g., for the treatment or diagnosis of a subject having a disease/disorder state, e.g., cancer.

In some embodiments, the cell state is selected from a state of health or disease or disorder, e.g., cancer, inflammation, metabolic disease and/or a state of differentiation, e.g., a less than fully differentiated cell, e.g., a stem cell, e.g., a cancer stem cell, or a precursor cell.

In some embodiments, evaluating includes acquiring knowledge of the selected enhancer component relevant to a cell state or the cell state.

In some embodiments, evaluating includes acquiring knowledge of the association, correlation, or role of the selected enhancer component a cell state or the cell state, e.g., cancer. In some embodiments, the knowledge includes knowledge of the effect of one or more of the following factors on the cell state, e.g., cancer, or on a parameter associated with the cell state, e.g., proliferation or cell death:

a) the effect of modulation of the level of gene expression, or of a gene product of the gene encoding the selected enhancer component;

b) the effect of gene targeting, e.g., by anti-sense or RNAi, of the gene encoding the selected enhancer component;

c) the effect of antagonizing the selected enhancer component, e.g., with an antibody or other binding ligand; or

d) the effect of a genetic event involving the gene encoding the selected enhancer component, e.g., a copy number alteration, e.g., amplification or deletion, a rearrangement, e.g., a translocation, deletion, insertion, or point mutations, or other mutations.

In some embodiments, the method includes acquiring the knowledge from a database, e.g., a public database.

In some embodiments, the method further includes evaluating whether the candidate super-enhancer component binds a super-enhancer.

In some embodiments, the method further includes evaluating whether down modulation of the candidate super-enhancer component disrupts (e.g., decreases) super-enhancer function.

In some embodiments, the method further includes evaluating whether down modulation of the candidate super-enhancer component disrupts (e.g., decreases) super-enhancer function more than it disrupts (e.g., decreases) enhancer function.

In some embodiments, the method includes determining if a candidate super-enhancer component is associated with a super-enhancer in samples from a plurality of subjects, e.g., from a plurality of cancer samples of the same or different types.

In some embodiments, the method further includes confirming, e.g., by performing experiments, one of the effects listed above.

In some embodiments, the method further includes evaluating the effect of knockdown by RNAi or expression of a dominant negative mutant of the target gene, on the cell state, e.g., in a cell-free, cellular, or animal model.

Aspects and embodiments are directed to methods of decreasing the activity, expression, or transcription of a component described in Table 1 or variant thereof, for example, to treat a disorder in a subject. In an embodiment, the methods described herein include administering to a subject an effective amount of a compound that decreases the activity, expression, or transcription of a component described in Table 1 or variant thereof, thereby treating a disorder in the subject. In an embodiment, the component of Table 1 is not one of KDM1, TCF3, MBD3, SMC1A, SMC1B, EP300, CHD4, HDAC1, NR5A2, MED1, SMARCA4, MYOD1, KLF4, BRD4, PRDM14, FOXO1, NIPBL, SMAD3, CHD7, SOX2, MED12, HDAC2, POU5F1, SIRT1, DOT1L, or CEBPA. Exemplary disorders are described herein, and include cancer, immunological disorders, and metabolic disorders.

An “effective amount” or “effective dose” of a compound (or composition containing such compound) generally refers to the amount sufficient to achieve a desired biological and/or pharmacological effect, e.g., when contacted with a cell in vitro or administered to a subject according to a selected administration form, route, and/or schedule. As will be appreciated by those of ordinary skill in the art, the absolute amount of a particular compound or composition that is effective may vary depending on such factors as the desired biological or pharmacological endpoint, the agent to be delivered, the target tissue, etc. Those of ordinary skill in the art will further understand that an “effective amount” may be contacted with cells or administered in a single dose, or through use of multiple doses, in various embodiments. It will be understood that compound, compounds, and compositions herein may be employed in an amount effective to achieve a desired biological and/or therapeutic effect.

The term “variant” encompasses “fragments” or “functional fragments” of a component of Table 1. A “fragment” is a continuous portion of a polypeptide or polynucleotide that is shorter than the original polypeptide or polynucleotide. In some embodiments a variant comprises or consists of a fragment. In some embodiments a fragment or variant is at least 20%, 30%, 40%, 50%, 60%, 70%, 80%, 90%, 95%, 96%, 97%, 98%, 99%, or more as long as the original polypeptide or polynucleotide. A fragment may be an N-terminal, C-terminal, or internal fragment. A functional fragment of a super-enhancer can have one or more of the following properties:

a) when associated with a gene, e.g., a gene with which it is normally associated, it provides at least 10, 20, 30, 40, 50, 60, 70, 80, or 90% of the level of expression as is seen with the intact component;

b) when associated with a gene, e.g., a gene with which it is normally associated, it provides at least 10, 20, 30, 40, 50, 60, 70, 80, or 90% of the level of binding of a component;

c) it is at least 10, 20, 30, 40, 5, 60, 70, 80 or 90%, as long as the component of which it is a functional fragment.

The term variant also encompasses “sequence variants,” e.g., “functional sequence variants,” of a component or fragment or functional fragment of a component. A functional sequence variant of a component can have one or more of the following properties:

a) it comprises sufficient nucleotide sequence homology or identity with a reference component, e.g., the component from which it is derived, that when associated with a gene, e.g., a gene with which the reference component is normally associated, it provides at least 10, 20, 30, 40, 50, 60, 70, 80, or 90% of the level of expression as is seen with the reference component;

b) when associated with a gene, e.g., a gene with which the reference component, e.g., the component from which it is derived, is normally associated, it provides at least 10, 20, 30, 40, 50, 60, 70, 80, or 90% of the level of binding of a component as is seen with the reference component;

c) it comprises at least 40, 50, 60, 70, 80, 90, 95, 97, or 99% sequence homology or identity with a reference component, e.g., the component from which it is derived.

In some embodiments a variant polypeptide comprises or consists of at least one domain of an original polypeptide. In some embodiments a variant polynucleotide hybridizes to an original polynucleotide under stringent conditions, e.g., high stringency conditions, for sequences of the length of the original polypeptide. In some embodiments a variant polypeptide or polynucleotide comprises or consists of a polypeptide or polynucleotide that is at least 50%, 60%, 70%, 80%, 90%, 95%, 96%, 97%, 98%, 99%, or more identical in sequence to the original polypeptide or polynucleotide over at least 20%, 30%, 40%, 50%, 60%, 70%, 80%, 90%, 95%, 96%, 97%, 98%, 99%, or 100% of the original polypeptide or polynucleotide. In some embodiments a variant polypeptide comprises or consists of a polypeptide that is at least 50%, 60%, 70%, 80%, 90%, 95%, 96%, 97%, 98%, 99%, or more identical in sequence to the original polypeptide over at least 20%, 30%, 40%, 50%, 60%, 70%, 80%, 90%, 95%, 96%, 97%, 98%, 99%, or 100% of the original polypeptide, with the proviso that, for purposes of computing percent identity, a conservative amino acid substitution is considered identical to the amino acid it replaces. In some embodiments a variant polypeptide comprises or consists of a polypeptide that is at least 50%, 60%, 70%, 80%, 90%, 95%, 96%, 97%, 98%, 99%, or more identical to the original polypeptide over at least 20%, 30%, 40%, 50%, 60%, 70%, 80%, 90%, 95%, 96%, 97%, 98%, 99%, or 100% of the original polypeptide, with the proviso that any one or more amino acid substitutions (up to the total number of such substitutions) may be restricted to conservative substitutions. In some embodiments a percent identity is measured over at least 100; 200; 300; 400; 500; 600; 700; 800; 900; 1,000; 1,200; 1,500; 2,000; 2,500; 3,000; 3,500; 4,000; 4,500; or 5,000 amino acids. In some embodiments the sequence of a variant polypeptide comprises or consists of a sequence that has N amino acid differences with respect to an original sequence, wherein N is any integer between 1 and 10 or between 1 and 20 or any integer up to 1%, 2%, 5%, or 10% of the number of amino acids in the original polypeptide, where an “amino acid difference” refers to a substitution, insertion, or deletion of an amino acid. In some embodiments a difference is a conservative substitution. Conservative substitutions may be made, e.g., on the basis of similarity in side chain size, polarity, charge, solubility, hydrophobicity, hydrophilicity and/or the amphipathic nature of the residues involved. In some embodiments, conservative substitutions may be made according to Table A, wherein amino acids in the same block in the second column and in the same line in the third column may be substituted for one another other in a conservative substitution. Certain conservative substitutions are substituting an amino acid in one row of the third column corresponding to a block in the second column with an amino acid from another row of the third column within the same block in the second column.

TABLE A

| | | |
|---|---|---|
| Aliphatic | Non-polar | G A P |
| | | I L V |
| | Polar-uncharged | C S T M |
| | | N Q |
| | Polar-charged | D E |
| | | K R |
| Aromatic | | H F W Y |

Aspects and embodiments are also directed to a computer-implemented method of evaluating a gene and/or an associated super-enhancer including selecting a gene associated with a super-enhancer, to provide a selected gene, evaluating, on a computer, the selected gene for contribution to a cell state, e.g., a state characterized by a disease or disorder (a disease/disorder state), and responsive to the evaluation, classifying the selected gene and/or associated super-enhancer.

Aspects and embodiments are also directed to a computer-implemented method of selecting a candidate super-enhancer component including selecting an enhancer component, e.g., a protein, e.g., a nuclear protein, that is associated with an enhancer, e.g., a transcription factor or chromatin modulator, to provide a selected enhancer component, e.g., a protein that contains a chromatin domain provided in Table 1, evaluating, on a computer, the selected enhancer component for contribution to a cell state, e.g., a state characterized by a disease or disorder (a disease/disorder state), and responsive to the evaluation, classifying the selected enhancer component as a candidate super-enhancer component. In an embodiment, the component of Table 1 is not one of KDM1, TCF3, MBD3, SMC1A, SMC1B, EP300, CHD4, HDAC1, NR5A2, MED1, SMARCA4, MYOD1, KLF4, BRD4, PRDM14, FOXO1, NIPBL, SMAD3, CHD7, SOX2, MED12, HDAC2, POU5F1, SIRT1, DOT1L, or CEBPA.

Aspects and embodiments are also directed to a system including a memory and a processing unit operative to select a gene associated with a super-enhancer, to provide a selected gene, evaluate the selected gene for contribution to a cell state, e.g., a state characterized by a disease or disorder (a disease/disorder state), and responsive to the evaluation, classify the selected gene and/or associated super-enhancer.

Aspects and embodiments are also directed to a system including a memory and a processing unit operative to select an enhancer component, e.g., a protein, e.g., a nuclear protein, that is associated with an enhancer, e.g., a transcription factor or chromatin modulator, to provide a selected enhancer component, e.g., a protein that contains a chromatin domain provided in Table 1, evaluate the selected enhancer component for contribution to a cell state, e.g., a state characterized by a disease or disorder (a disease/disorder state), and responsive to the evaluation, classify the selected enhancer component as a candidate super-enhancer component. In an embodiment, the component of Table 1 is not one of KDM1, TCF3, MBD3, SMC1A, SMC1B, EP300, CHD4, HDAC1, NR5A2, MED1, SMARCA4, MYOD1, KLF4, BRD4, PRDM14, FOXO1, NIPBL, SMAD3, CHD7, SOX2, MED12, HDAC2, POU5F1, SIRT1, DOT1L, or CEBPA.

Aspects and embodiments are also directed to a computer-readable medium comprising computer-executable instructions that, when executed on a processor of a computer, perform a method for evaluating a gene and/or an associate super-enhancer, comprising acts of selecting a gene associated with a super-enhancer, to provide a selected gene, evaluating, on a computer, the selected gene for contribution to a cell state, e.g., a state characterized by a disease or disorder (a disease/disorder state), and responsive to the evaluation, classifying the selected gene and/or associated super-enhancer.

Aspects and embodiments are also directed to a computer-readable medium comprising computer-executable instructions that, when executed on a processor of a computer, perform a method for evaluating a candidate super-enhancer component, comprising acts of selecting an enhancer component, e.g., a protein, e.g., a nuclear protein, that is associated with an enhancer, e.g., a transcription factor or chromatin modulator, to provide a selected enhancer component, e.g., a protein that contains a chromatin domain provided in Table 1, evaluating, on a computer, the selected enhancer component for contribution to a cell state, e.g., a state characterized by a disease or disorder (a disease/disorder state), and responsive to the evaluation, classifying the selected enhancer component as a candidate super-enhancer component. In an embodiment, the component of Table 1 is not one of KDM1, TCF3, MBD3, SMC1A, SMC1B, EP300, CHD4, HDAC1, NR5A2, MED1, SMARCA4, MYOD1, KLF4, BRD4, PRDM14, FOXO1, NIPBL, SMAD3, CHD7, SOX2, MED12, HDAC2, POU5F1, SIRT1, DOT1L, or CEBPA.

Still other aspects, embodiments, and advantages of these exemplary aspects and embodiments are discussed in detail below. Embodiments disclosed herein may be combined with other embodiments in any manner consistent with at least one of the principles disclosed herein, and references to “an embodiment,” “some embodiments,” “an alternate embodiment,” “various embodiments,” “one embodiment” or the like are not necessarily mutually exclusive and are intended to indicate that a particular feature, structure, or characteristic described may be included in at least one embodiment. The appearances of such terms herein are not necessarily all referring to the same embodiment.

### BRIEF DESCRIPTION OF THE DRAWINGS

Various aspects of at least one embodiment are discussed below with reference to the accompanying figures, which are not intended to be drawn to scale. The figures are included to provide illustration and a further understanding of the various aspects and embodiments, and are incorporated in and constitute a part of this specification, but are not intended as a definition of the limits of the disclosure. In the figures, each identical or nearly identical component that is illustrated in various figures is represented by a like numeral. For purposes of clarity, not every component may be labeled in every figure. In the figures:

FIG. 1 illustrates an exemplary computer system upon which various aspects of the present embodiments may be implemented;

FIG. 2 illustrates an exemplary embodiment of a system for evaluating genes;

FIG. 3 is a flow chart illustrating an exemplary process of evaluating genes;

FIG. 4 is exemplary process of evaluating an enhancer component; and

FIG. 5 is a table, Table 1, listing components useful in the methods described herein.

### DETAILED DESCRIPTION

In some embodiments, genes can be selected based on an association with one or more super-enhancers. The selected genes can be further evaluated and classified as a candidate target gene, for example, for treatment or diagnosis of a subject having a disease or disorder state, such as cancer. The evaluation can be for contribution to a cell state, such as a state characterized by a disease or disorder. In some embodiments, evaluation can include searching one or more databases for knowledge of association, correlation, and/or role of the gene to the contribution to the cell state. For example, databases can be searched to determine the effect of modulation, the effect of gene targeting, the effect of a synthetic lethal screen, the effect of a genetic event involving the gene, the expression or hybrid capture sequencing data, the effect of overexpressing a dominant negative mutant of the selected gene, and/or the effect of reducing the copy number of the gene. In some embodiments, one or more computer systems can be used to select, evaluate, and/or classify the genes.

In some embodiments, candidate super-enhancer components can be selected. Super-enhancer components can include proteins that are associated with super-enhancers. The super-enhancer components can be selected by selecting enhancer components associated with enhancers. The selected enhancer components can be evaluated for contribution to a cell state, such as a state characterized by a disease or disorder. Based on the evaluation, the selected enhancer component can be classified as a candidate super-enhancer component. In some embodiments, evaluation can include searching one or more databases for knowledge of association, correlation, and/or role of the gene to the contribution to the cell state. For example, databases can be searched to determine the effect of modulation of the level of gene expression, the effect of gene targeting, the effect of antagonizing the selected enhancer component, and/or the effect of a genetic event involving the gene encoding the selected enhancer component.

It is to be appreciated that embodiments of the methods and apparatuses discussed herein are not limited in application to the details of construction and the arrangement of components set forth in the following description or illustrated in the accompanying drawings. The methods and apparatuses are capable of implementation in other embodiments and of being practiced or of being carried out in various ways. Examples of specific implementations are provided herein for illustrative purposes only and are not intended to be limiting. In particular, acts, elements, and features discussed in connection with any one or more embodiments are not intended to be excluded from a similar role in any other embodiment.

Also, the phraseology and terminology used herein is for the purpose of description and should not be regarded as limiting. Any references to embodiments or elements or acts of the systems and methods herein referred to in the singular may also embrace embodiments including a plurality of these elements, and any references in plural to any embodiment or element or act herein may also embrace embodiments including only a single element. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. Any references to front and back, left and right, top and bottom, upper and lower, and vertical and horizontal are intended for convenience of description, not to limit the present systems and methods or their components to any one positional or spatial orientation.

Methods of Treatment

Embodiments described herein include methods of treatment. For example, inhibiting (e.g., fully or partially) a component described in Table 1 can reduce the activity or disrupt the structure of a super-enhancer. Super-enhancers have been discovered to control genes that can be required for maintenance of cell identity (e.g., embryonic stem cell identity) or maintenance of a disease state (e.g., cancer, Alzheimer's disease, Type 1 diabetes, and systemic lupus erythematosus). Accordingly, decreasing the activity, expression, or transcription of a component described in Table 1 or variant thereof can be used to treat a disorder in a subject, for example by reducing the activity or disrupting the structure of a super-enhancer. In an embodiment, the activity, expression, or transcription of a component described in Table 1 or variant thereof can be decreased by administering a compound to a subject that decreases the, activity, expression, or transcription of the component described in Table 1 or variant thereof. Complete inhibition of a component of Table 1 or complete reduction in the activity or disruption of the structure of a super-enhancer is not necessary for effective treatment of a subject.

The methods described herein can be applied to animals such as humans or to cells. For example, the compounds used in the methods described herein can be administered to cells in culture, e.g. in vitro or ex vivo, or to a subject, e.g., in vivo, to treat a variety of diseases or disorders (e.g., cancer (for example solid tumors), autoimmune disorders, cardiovascular disorders, inflammatory disorders, disorders of the central nervous system, metabolic disorders, infectious diseases, etc.).

Thus, in another aspect, the invention features, a method of treating cancer (for example a solid tumor), an autoimmune disorder or inflammatory disorder, a cardiovascular disorder, a metabolic disorder, a disorder of the central nervous system, or an infectious disease in a subject. The method comprises administering an effective amount of a compound described herein to a subject to thereby treat the disease or disorder. Exemplary diseases and disorders are described herein.

### Cancer

Accordingly, in another aspect, the invention features, a method of treating cancer (for example a solid tumor). The method comprises administering an effective amount of a compound described herein to thereby treat the disease or disorder.

In embodiments the compounds are used to treat proliferative disorders, e.g., treating a tumor and metastases thereof wherein the tumor or metastases thereof is a cancer described herein.

In embodiments, the proliferative disorder is a solid tumor, a soft tissue tumor or a liquid tumor. Exemplary solid tumors include malignancies (e.g., sarcomas and carcinomas (e.g., adenocarcinoma or squamous cell carcinoma)) of the various organ systems, such as those of brain, lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non-small cell carcinoma of the lung, and cancer of the small intestine. In embodiments the method comprises evaluating or treating soft tissue tumors such as those of the tendons, muscles or fat, and liquid tumors.

In embodiment the cancer is any cancer, for example those described by the National Cancer Institute. The cancer can be a carcinoma, a sarcoma, a myeloma, a leukemia, a lymphoma or a mixed type. Exemplary cancers described by the National Cancer Institute include:

Digestive/gastrointestinal cancers such as anal cancer; bile duct cancer; extrahepatic bile duct cancer; appendix cancer; carcinoid tumor, gastrointestinal cancer; colon cancer; colorectal cancer including childhood colorectal cancer; esophageal cancer including childhood esophageal cancer; gallbladder cancer; gastric (stomach) cancer including childhood gastric (stomach) cancer; hepatocellular (liver) cancer including adult (primary) hepatocellular (liver) cancer and childhood (primary) hepatocellular (liver) cancer; pancreatic cancer including childhood pancreatic cancer; sarcoma, rhabdomyosarcoma; islet cell pancreatic cancer; rectal cancer; and small intestine cancer;

Endocrine cancers such as islet cell carcinoma (endocrine pancreas); adrenocortical carcinoma including childhood adrenocortical carcinoma; gastrointestinal carcinoid tumor; parathyroid cancer; pheochromocytoma; pituitary tumor; thyroid cancer including childhood thyroid cancer; childhood multiple endocrine neoplasia syndrome; and childhood carcinoid tumor;

Eye cancers such as intraocular melanoma; and retinoblastoma;

Musculoskeletal cancers such as Ewing's family of tumors; osteosarcoma/malignant fibrous histiocytoma of the bone; childhood rhabdomyosarcoma; soft tissue sarcoma including adult and childhood soft tissue sarcoma; clear cell sarcoma of tendon sheaths; and uterine sarcoma;

Breast cancer such as breast cancer including childhood and male breast cancer and pregnancy;

Neurologic cancers such as childhood brain stem glioma; brain tumor; childhood cerebellar astrocytoma; childhood cerebral astrocytoma/malignant glioma; childhood ependymoma; childhood medulloblastoma; childhood pineal and supratentorial primitive neuroectodermal tumors; childhood visual pathway and hypothalamic glioma; other childhood brain cancers; adrenocortical carcinoma; central nervous system lymphoma, primary; childhood cerebellar astrocytoma; neuroblastoma; craniopharyngioma; spinal cord tumors; central nervous system atypical teratoid/rhabdoid tumor; central nervous system embryonal tumors; and childhood supratentorial primitive neuroectodermal tumors and pituitary tumor;

Genitourinary cancers such as bladder cancer including childhood bladder cancer; renal cell (kidney) cancer; ovarian cancer including childhood ovarian cancer; ovarian epithelial cancer; ovarian low malignant potential tumor; penile cancer; prostate cancer; renal cell cancer including childhood renal cell cancer; renal pelvis and ureter, transitional cell cancer; testicular cancer; urethral cancer; vaginal cancer; vulvar cancer; cervical cancer; Wilms tumor and other childhood kidney tumors; endometrial cancer; and gestational trophoblastic tumor;

Germ cell cancers such as childhood extracranial germ cell tumor; extragonadal germ cell tumor; ovarian germ cell tumor; and testicular cancer;

Head and neck cancers such as lip and oral cavity cancer; oral cancer including childhood oral cancer; hypopharyngeal cancer; laryngeal cancer including childhood laryngeal cancer; metastatic squamous neck cancer with occult primary; mouth cancer; nasal cavity and paranasal sinus cancer; nasopharyngeal cancer including childhood nasopharyngeal cancer; oropharyngeal cancer; parathyroid cancer; pharyngeal cancer; salivary gland cancer including childhood salivary gland cancer; throat cancer; and thyroid cancer;

Hematologic/blood cell cancers such as a leukemia (e.g., acute lymphoblastic leukemia including adult and childhood acute lymphoblastic leukemia; acute myeloid leukemia including adult and childhood acute myeloid leukemia; chronic lymphocytic leukemia; chronic myelogenous leukemia; and hairy cell leukemia); a lymphoma (e.g., AIDS-related lymphoma; cutaneous T-cell lymphoma; Hodgkin's lymphoma including adult and childhood Hodgkin's lymphoma and Hodgkin's lymphoma during pregnancy; non-Hodgkin's lymphoma including adult and childhood non-Hodgkin's lymphoma and non-Hodgkin's lymphoma during pregnancy; mycosis fungoides; Sézary syndrome; Waldenstrom's macroglobulinemia; and primary central nervous system lymphoma); and other hematologic cancers (e.g., chronic myeloproliferative disorders; multiple myeloma/plasma cell neoplasm; myelodysplastic syndromes; and myelodysplastic/myeloproliferative disorders);

Lung cancer such as non-small cell lung cancer; and small cell lung cancer;

Respiratory cancers such as malignant mesothelioma, adult; malignant mesothelioma, childhood; malignant thymoma; childhood thymoma; thymic carcinoma; bronchial adenomas/carcinoids including childhood bronchial adenomas/carcinoids; pleuropulmonary blastoma; non-small cell lung cancer; and small cell lung cancer;

Skin cancers such as Kaposi's sarcoma; Merkel cell carcinoma; melanoma; and childhood skin cancer;

AIDS-related malignancies;

Other childhood cancers, unusual cancers of childhood and cancers of unknown primary site;

and metastases of the aforementioned cancers can also be treated or prevented in accordance with the methods described herein.

### Inflammation and Autoimmune Disease

In another aspect, the invention features, a method of treating inflammation or an autoimmune disease. The method comprises administering an effective amount of a compound described herein to thereby treat the disease or disorder.

Exemplary inflammatory conditions include, for example, multiple sclerosis, rheumatoid arthritis, psoriatic arthritis, degenerative joint disease, spondouloarthropathies, gouty arthritis, systemic lupus erythematosus, juvenile arthritis, rheumatoid arthritis, osteoarthritis, osteoporosis, diabetes (e.g., insulin dependent diabetes mellitus or juvenile onset diabetes), menstrual cramps, cystic fibrosis, inflammatory bowel disease, irritable bowel syndrome, Crohn's disease, mucous colitis, ulcerative colitis, gastritis, esophagitis, pancreatitis, peritonitis, Alzheimer's disease, shock, ankylosing spondylitis, gastritis, conjunctivitis, pancreatis (acute or chronic), multiple organ injury syndrome (e.g., secondary to septicemia or trauma), myocardial infarction, atherosclerosis, stroke, reperfusion injury (e.g., due to cardiopulmonary bypass or kidney dialysis), acute glomerulonephritis, vasculitis, thermal injury (i.e., sunburn), necrotizing enterocolitis, granulocyte transfusion associated syndrome, and/or Sjogren's syndrome. Exemplary inflammatory conditions of the skin include, for example, eczema, atopic dermatitis, contact dermatitis, urticaria, schleroderma, psoriasis, and dermatosis with acute inflammatory components.

In another embodiment, a compound described herein may be used to treat or prevent allergies and respiratory conditions, including asthma, bronchitis, pulmonary fibrosis, allergic rhinitis, oxygen toxicity, emphysema, chronic bronchitis, acute respiratory distress syndrome, and any chronic obstructive pulmonary disease (COPD). The polymer-agent conjugate, particle or composition may be used to treat chronic hepatitis infection, including hepatitis B and hepatitis C.

In embodiments a compound described herein may be used to treat autoimmune diseases and/or inflammation associated with autoimmune diseases such as organ-tissue autoimmune diseases (e.g., Raynaud's syndrome), scleroderma, myasthenia gravis, transplant rejection, endotoxin shock, sepsis, psoriasis, eczema, dermatitis, multiple sclerosis, autoimmune thyroiditis, uveitis, systemic lupus erythematosis, Addison's disease, autoimmune polyglandular disease (also known as autoimmune polyglandular syndrome), and Grave's disease.

### Cardiovascular Disease

In another aspect, the invention features, a method of treating a cardiovascular disease. The method comprises administering an effective amount of a compound described herein to thereby treat the disease or disorder.

Cardiovascular diseases that can be treated or prevented using a compound described herein include cardiomyopathy or myocarditis; such as idiopathic cardiomyopathy, metabolic cardiomyopathy, alcoholic cardiomyopathy, drug-induced cardiomyopathy, ischemic cardiomyopathy, and hypertensive cardiomyopathy. Also treatable are atheromatous disorders of the major blood vessels (macrovascular disease) such as the aorta, the coronary arteries, the carotid arteries, the cerebrovascular arteries, the renal arteries, the iliac arteries, the femoral arteries, and the popliteal arteries. In embodiments other vascular diseases that can be treated or prevented include those related to platelet aggregation, the retinal arterioles, the glomerular arterioles, the vasa nervorum, cardiac arterioles, and associated capillary beds of the eye, the kidney, the heart, and the central and peripheral nervous systems. The compounds described herein may also be used for increasing HDL levels in plasma of an individual.

Metabolic Disorders

In particular, the disclosure features the use of a compound described herein for the treatment or prevention of a metabolic disorder in a subject, e.g., a human subject. The term “metabolic disorder” includes a disorder, disease or condition which is caused or characterized by an abnormal metabolism (i.e., the chemical changes in living cells by which enemy is provided for vital processes and activities) in a subject. Examples of disorders include obesity, diabetes, a co-morbidity of obesity, and an obesity related disorder. The subject to whom the polymer-agent, particle or composition is administered may be overweight or obese. Alternatively, or in addition, the subject may be diabetic, for example having insulin resistance or glucose intolerance, or both. The subject may have diabetes mellitus, for example, the subject may have Type II diabetes. The subject may be overweight or obese and have diabetes mellitus, for example, Type II diabetes.

In addition, or alternatively, the subject may have, or may be at risk of having, a disorder in which obesity or being overweight is a risk factor. As used herein, “obesity” refers to a body mass index (BMI) of 30 kg/m² or more (National Institute of Health, Clinical Guidelines on the Identification, Evaluation, and Treatment of Overweight and Obesity in Adults (1998)). However, the present invention is also intended to include a disease, disorder, or condition that is characterized by a body mass index (BMI) of 25 kg/m² or more, 26 kg/m² or more, 27 kg/m² or more, 28 kg/m² or more, 29 kg/m² or more, 29.5 kg/m² or more, all of which are typically referred to as overweight (National Institute of Health, Clinical Guidelines on the Identification, Evaluation, and Treatment of Overweight and Obesity in Adults (1998)). Such disorders include, but are not limited to, cardiovascular disease, for example hypertension, atherosclerosis, congestive heart failure, and dyslipidemia; stroke; gallbladder disease; osteoarthritis; sleep apnea; reproductive disorders for example, polycystic ovarian syndrome; cancers, for example breast, prostate, colon, endometrial, kidney, and esophagus cancer; varicose veins; acanthosis nigricans; eczema; exercise intolerance; insulin resistance; hypertension; hypercholesterolemia; cholithiasis; osteoarthritis; orthopedic injury; insulin resistance, for example, type 2 diabetes and syndrome X; metabolic syndrome; and thromboembolic disease (see Kopelman (2000), Nature 404:635-43; Rissanen et al., British Med. J. 301, 835, 1990).

Other disorders associated with obesity include depression, anxiety, panic attacks, migraine headaches, PMS, chronic pain states, fibromyalgia, insomnia, impulsivity, obsessive-compulsive disorder, irritable bowel syndrome (IBS), and myoclonus. Furthermore, obesity is a recognized risk factor for increased incidence of complications of general anesthesia. (See e.g., Kopelman, Nature 404:635-43, 2000). In general, obesity reduces life span and carries a serious risk of co-morbidities such as those listed above. Other diseases or disorders associated with obesity are birth defects, maternal obesity being associated with increased incidence of neural tube defects, carpal tunnel syndrome (CTS); chronic venous insufficiency (CVI); daytime sleepiness; deep vein thrombosis (DVT); end stage renal disease (ESRD); gout; heat disorders; impaired immune response; impaired respiratory function; infertility; liver disease; lower back pain; obstetric and gynecologic complications; pancreatititis; as well as abdominal hernias; acanthosis nigricans; endocrine abnormalities; chronic hypoxia and hypercapnia; dermatological effects; elephantitis; gastroesophageal reflux; heel spurs; lower extremity edema; mammegaly which causes considerable problems such as bra strap pain, skin damage, cervical pain, chronic odors and infections in the skin folds under the breasts, etc.; large anterior abdominal wall masses, for example abdominal panniculitis with frequent panniculitis, impeding walking, causing frequent infections, odors, clothing difficulties, lower back pain; musculoskeletal disease; pseudo tumor cerebri (or benign intracranial hypertension), and sliding hiatil hernia.

Conditions or disorders associated with increased caloric intake include, but are not limited to, insulin resistance, glucose intolerance, obesity, diabetes, including type 2 diabetes, eating disorders, insulin-resistance syndromes, metabolic syndrome X, and Alzheimer's disease.

Central Nervous System Disorders

Provided herein are methods of treating central nervous system disorders in a subject, e.g., a human subject, that comprise administering to said subject a therapeutically effective amount of a compound as disclosed herein. Examples of central nervous system disorders include, but are not limited to: a myelopathy; an encephalopathy; central nervous system (CNS) infection; encephalitis (e.g., viral encephalitis, bacterial encephalitis, parasitic encephalitis); meningitis (e.g., spinal meningitis, bacterial meningitis, viral meningitis, fungal meningitis); neurodegenerative diseases (e.g., Huntington's disease; Alzheimer's disease; Parkinson's disease; multiple sclerosis; amyotrophic lateral sclerosis; traumatic brain injury); mental health disorder (e.g., schizophrenia, depression, dementia); pain and addiction disorders; brain tumors (e.g., intra-axial tumors, extra-axial tumors); adult brain tumors (e.g., glioma, glioblastoma); pediatric brain tumors (e.g., medulloblastoma); cognitive impairment; genetic disorders (e.g., Huntington's disease, neurofibromatosis type 1, neurofibromatosis type 2, Tay-Sachs disease, tuberous sclerosis); headache (e.g., tension headache; migraine headache, cluster headache, meningitis headache, cerebral aneurysm and subarachnoid hemorrhage headache, brain tumor headache); stroke (e.g., cerebral ischemia or cerebral infarction, transient ischemic attack, hemorrhagic (e.g., aneurysmal subarachnoid hemorrhage, hypertensive hemorrhage, other sudden hemorrhage)); epilepsy; spinal disease (e.g., degenerative spinal disease (e.g., herniated disc disease, spinal stenosis, and spinal instability), traumatic spine disease; spinal cord trauma; spinal tumors; hydrocephalus (e.g., communicating or non-obstructive hydrocephalus, non-communicating or obstructive hydrocephalus, adult hydrocephalus, pediatric hydrocephalus, normal pressure hydrocephalus, aqueductal stenosis, tumor associated hydrocephalus, pseudotumor cerebri); CNS vasculitis (e.g., primary angiitis of the central nervous system, benign angiopathy of the central nervous system; Arnold Chiari malformation; neuroAlDS; retinal disorders (e.g., age-related macular degeneration, wet age-related macular degeneration, myopic macular degeneration, retinitis pigmentosa, proliferative retinopathies); inner ear disorders; tropical spastic paraparesis; arachnoid cysts; locked-in syndrome; Tourette's syndrome; adhesive arachnoiditis; altered consciousness; autonomic neuropathy; benign essential tremor; brain anomalies; cauda equine syndrome with neurogenic bladder; cerebral edema; cerebral spasticity; cerebral vascular disorder; and Guillain-Barre syndrome.

### Neurological Deficits

Methods can be used to treat neurological deficits due to neurodegeneration in the brain of a subject, e.g., a human subject. The method can include administering a compound described herein to the subject. As used herein, the phrase “neurological deficits” includes an impairment or absence of a normal neurological function or presence of an abnormal neurological function. Neurodegeneration of the brain can be the result of disease, injury, and/or aging. As used herein, neurodegeneration includes morphological and/or functional abnormality of a neural cell or a population of neural cells. Non-limiting examples of morphological and functional abnormalities include physical deterioration and/or death of neural cells, abnormal growth patterns of neural cells, abnormalities in the physical connection between neural cells, under- or over production of a substance or substances, e.g., a neurotransmitter, by neural cells, failure of neural cells to produce a substance or substances which it normally produces, production of substances, e.g., neurotransmitters, and/or transmission of electrical impulses in abnormal patterns or at abnormal times. Neurodegeneration can occur in any area of the brain of a subject and is seen with many disorders including, for example, head trauma, stroke, ALS, multiple sclerosis, Huntington's disease, Parkinson's disease, and Alzheimer's disease.

Bacterial Injections

Described herein are methods for treating bacterial infections. Examples of bacterial organisms that may be controlled by the compounds described herein include, but are not limited to the following organisms: Streptococcus pneumoniae, Streptococcus pyogenes, Enterococcus faecalis, Enterococcus faeciuin, Klebsiella pneumoniae, Enterobacter sps. Proteus sps. Pseudomonas aeruginosa, E. coli, Serratia inarcesens, Staphylococcus aureus, Coag. Neg. Staph, Haemophilus infuenzae, Bacillus anthracis, Mycoplasma pneumoniae, Moraxella catarralis, Chlamydia pneumoniae, Legionella pneumophila, Mycobacterium tuberculosis, Helicobacter pylori, Staphylococcus saprophyticus, and Staphylococcus epidermidis.

A compound described herein can be useful for the treatment of a gram positive infection or a gram negative infection. A compound described herein can also be useful in the treatment of nosocomial or non-nosocomial infections. Examples of nosocomial infection uses include, but are not limited to, urinary tract infections, pneumonia, surgical wound infections, bone and joint infections, and bloodstream infections. Examples of non-nosocomial uses include but are not limited to urinary tract infections, pneumonia, prostatitis, skin and soft tissue infections, bone and joint infections, intra-abdominal infections, meningitis, brain abscess, infectious diarrhea and gastrointestinal infections, surgical prophylaxis, and therapy for febrile neutropenic patients. The term “non-nosocomial infections” is also referred to as community acquired infections.

Compounds

Compounds of use in various embodiments of the invention can comprise, e.g., small molecules, peptides, polypeptides, nucleic acids, oligonucleotides, etc. Certain non-limiting examples are presented below.

In an embodiment, the compound is a small molecule. A small molecule is often an organic compound having a molecular weight equal to or less than 2.0 kD, e.g., equal to or less than 1.5 kD, e.g., equal to or less than 1 kD, e.g., equal to or less than 500 daltons and usually multiple carbon-carbon bonds. Small molecules often comprise one or more functional groups that mediate structural interactions with proteins, e.g., hydrogen bonding, and typically include at least an amine, carbonyl, hydroxyl or carboxyl group, and in some embodiments at least two of the functional chemical groups. A small molecule may comprise cyclic carbon or heterocyclic structures and/or aromatic or polyaromatic structures substituted with one or more chemical functional groups and/or heteroatoms. In some embodiments a small molecule satisfies at least 3, 4, or all criteria of Lipinski's “Rule of Five”. In some embodiments, a compound is cell-permeable, e.g., within the range of typical compounds that act intracellularly, e.g., within mammalian cells. In some embodiments, the IC50 of a compound, e.g., a small molecule, for a target to be inhibited is less than or equal to about 5 nM, 10 nM, 50 nM, 100 nM, 500 nM, 1 μM, 10 μM, 50 μM, or 100 μM,

In an embodiment, the compound is a nucleic acid. Nucleic acids, e.g., oligonucleotides (which typically refers to short nucleic acids, e.g., 50 nucleotides in length or less), the invention contemplates use of oligonucleotides that are single-stranded, double-stranded (ds), blunt-ended, or double-stranded with overhangs, in various embodiments of the invention. The full spectrum of modifications (e.g., nucleoside and/or backbone modifications), non-standard nucleotides, delivery vehicles and systems, etc., known in the art as being useful in the context of siRNA or antisense-based molecules for research or therapeutic purposes is contemplated for use in various embodiments of the instant invention. In some embodiments a compound is an RNAi agent, antisense oligonucleotide, or aptamer. The term “RNAi agent” encompasses nucleic acids that can be used to achieve RNA silencing in mammalian cells. As used herein RNA silencing, also termed RNA interference (RNAi), encompasses processes in which sequence-specific silencing of gene expression is effected by an RNA-induced silencing complex (RISC) that has a short RNA strand incorporated therein, which strand directs or “guides” sequence-specific degradation or translational repression of mRNA to which it has complementarity. The complementarity between the short RNA and mRNA need not be perfect (100%) but need only be sufficient to result in inhibition of gene expression. For example, the degree of complementarity and/or the characteristics of the structure formed by hybridization of the mRNA and the short RNA strand can be such that the strand can (i) guide cleavage of the mRNA in the RNA-induced silencing complex (RISC) and/or (ii) cause translational repression of the mRNA by RISC. The short RNA is often incorporated into RISC as part of a short double-stranded RNA (dsRNA). RNAi may be achieved artificially in eukaryotic, e.g., mammalian, cells in a variety of ways. For example, RNAi may be achieved by introducing an appropriate short double-stranded nucleic acid into the cells or expressing in the cells a nucleic acid that is processed intracellularly to yield such short dsRNA. Exemplary RNAi agents are a short hairpin RNA (shRNA), a short interfering RNA (siRNA), micrRNA (miRNA) and a miRNA precursor. siRNAs typically comprise two separate nucleic acid strands that are hybridized to each other to form a duplex. They can be synthesized in vitro, e.g., using standard nucleic acid synthesis techniques. A nucleic acid may contain one or more non-standard nucleotides, modified nucleosides (e.g., having modified bases and/or sugars) or nucleotide analogs, and/or have a modified backbone. Any modification or analog recognized in the art as being useful for RNAi, aptamers, antisense molecules or other uses of oligonucleotides can be used. Some modifications result in increased stability, cell uptake, potency, etc. Exemplary compound can comprise morpholinos or locked nucleic acids. In some embodiments the nucleic acid differs from standard RNA or DNA by having partial or complete 2′-0-methylation or 2′-0-methoxyethyl modification of sugar, phosphorothioate backbone, and/or a cholesterol-moiety at the 3′-end. In certain embodiments the siRNA or shRNA comprises a duplex about 19 nucleotides in length, wherein one or both strands has a 3′ overhang of 1-5 nucleotides in length (e.g., 2 nucleotides), which may be composed of deoxyribonucleo tides. shRNA comprise a single nucleic acid strand that contains two complementary portions separated by a predominantly non-self-complementary region. The complementary portions hybridize to form a duplex structure and the non-self-complementary region forms a loop connecting the 3′ end of one strand of the duplex and the 5′ end of the other strand. shRNAs can undergo intracellular processing to generate siRNAs. In certain embodiments the term “RNAi agent” also encompasses vectors, e.g., expression vectors, that comprise templates for transcription of an siRNA (e.g., as two separate strands that can hybridize), shRNA, or microRNA precursor, and can be used to introduce such template into mammalian cells and result in transient or stable expression thereof.

In some embodiments an RNAi agent, aptamer, antisense oligonucleotide, other nucleic acid, peptide, polypeptide, or small molecule is physically associated with a moiety that increases cell uptake, such as a cell-penetrating peptide, or a delivery agent. In some embodiments a delivery agent at least in part protects the compound from degradation, metabolism, or elimination from the body (e.g., increases the half-life). A variety of compositions and methods can be used to deliver agents to cells in vitro or in vivo. For example, compounds can be attached to a polyalkylene oxide, e.g., polyethylene glycol (PEG) or a derivative thereof, or incorporated into or attached to various types of molecules or particles such as liposomes, lipoplexes, or polymer-based particles, e.g., microparticles or nanoparticles composed at least in part of one or more biocompatible polymers or copolymers comprising poly(lactide-glycolide), copolyoxalates, polycaprolactones, polyesterar des, polyorthoesters, polyhydroxybutyric acid, and/or polyanhydrides.

In some embodiments, an agent comprises a polypeptide. A “polypeptide” refers to a polymer of amino acids linked by peptide bonds. A protein is a molecule comprising one or more polypeptides. A peptide is a relatively short polypeptide, typically between about 2 and 100 amino acids (aa) in length, e.g., between 4 and 60 aa; between 8 and 40 aa; between 10 and 30 aa. The terms “protein”, “polypeptide”, and “peptide” may be used interchangeably. In general, a polypeptide may contain only standard amino acids or may comprise one or more non-standard amino acids (which may be naturally occurring or non-naturally occurring amino acids) and/or amino acid analogs in various embodiments. A “standard amino acid” is any of the 20 L-amino acids that are commonly utilized in the synthesis of proteins by mammals and are encoded by the genetic code. A “non-standard amino acid” is an amino acid that is not commonly utilized in the synthesis of proteins by mammals. Non-standard amino acids include naturally occurring amino acids (other than the 20 standard amino acids) and non-naturally occurring amino acids. In some embodiments, a non-standard, naturally occurring amino acid is found in mammals. For example, ornithine, citrulline, and homocysteine are naturally occurring non-standard amino acids that have important roles in mammalian metabolism. Exemplary non-standard amino acids include, e.g., singly or multiply halogenated (e.g., fluorinated) amino acids, D-amino acids, homo-amino acids, N-alkyl amino acids (other than proline), dehydroamino acids, aromatic amino acids (other than histidine, phenylalanine, tyrosine and tryptophan), and α,α disubstituted amino acids. An amino acid, e.g., one or more of the amino acids in a polypeptide, may be modified, for example, by addition, e.g., covalent linkage, of a moiety such as an alkyl group, an alkanoyl group, a carbohydrate group, a phosphate group, a lipid, a polysaccharide, a halogen, a linker for conjugation, a protecting group, etc. Modifications may occur anywhere in a polypeptide, e.g., the peptide backbone, the amino acid side-chains and the amino or carboxyl termini. A given polypeptide may contain many types of modifications. Polypeptides may be branched or they may be cyclic, with or without branching. Polypeptides may be conjugated with, encapsulated by, or embedded within a polymer or polymeric matrix, dendrimer, nanoparticle, microparticle, liposome, or the like. Modification may occur prior to or after an amino acid is incorporated into a polypeptide in various embodiments. Polypeptides may, for example, be purified from natural sources, produced in vitro or in vivo in suitable expression systems using recombinant DNA technology (e.g., by recombinant host cells or in transgenic animals or plants), synthesized through chemical means such as conventional solid phase peptide synthesis, and/or methods involving chemical ligation of synthesized peptides (see, e.g., Kent, S., J Pept Sci., 9(9):574-93, 2003 or U.S. Pub. No. 20040115774), or any combination of the foregoing.

One of ordinary skill in the art will understand that a protein may be composed of a single amino acid chain or multiple chains associated covalently or noncovalently.

In some embodiments a compound comprises an antibody. The term “antibody” encompasses immunoglobulins and derivatives thereof containing an immunoglobulin domain capable of binding to an antigen. An antibody can originate from any mammalian or avian species, e.g., human, rodent (e.g., mouse, rabbit), goat, chicken, etc., or can be generated using, e.g., phage display. The antibody may be a member of any immunoglobulin class, e.g., IgG IgM, IgA, IgD, IgE, or subclasses thereof such as IgG1, IgG2, etc. In various embodiments of the invention “antibody” refers to an antibody fragment such as an Fab′, F(ab′)2, scFv (single-chain variable) or other fragment that retains an antigen binding site, or a recombinantly produced scFv fragment, including recombinantly produced fragments. An antibody can be monovalent, bivalent or multivalent in various embodiments. The antibody may be a chimeric or “humanized” antibody, which can be generated using methods known in the art. An antibody may be polyclonal or monoclonal, though monoclonal antibodies may be preferred. Methods for producing antibodies that specifically bind to virtually any molecule of interest are known in the art. In some aspects the antibody is an intrabody, which may be expressed intracellularly. In some embodiments a compound comprises a single-chain antibody and a protein transduction domain (e.g., as a fusion polypeptide).

Also described herein are compositions comprising a compound described herein and a pharmaceutically acceptable carrier, diluent, or excipient. Therapeutic formulations of the compounds described herein can be prepared having the desired degree of purity with optional pharmaceutically acceptable carriers, excipients or stabilizers (REMINGTON'S PHARMACEUTICAL SCIENCES (A. Osol ed. 1980), which is hereby incorporated by reference in its entirety), in the form of lyophilized formulations or aqueous solutions. Acceptable carriers, excipients, or stabilizers are nontoxic to recipients at the dosages and concentrations employed, and include buffers such as acetate, Tris-phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; metal complexes (e.g., Zn-protein complexes); and/or non-ionic surfactants such as TWEEN®, PLURONICS® or polyethylene glycol (PEG).

Example Computer System

Various aspects and functions described herein in accord with the present disclosure may be implemented as hardware, software, or a combination of hardware and software on one or more computer systems. There are many examples of computer systems currently in use. Some examples include, among others, network appliances, personal computers, workstations, mainframes, networked clients, servers, media servers, application servers, database servers, web servers, and virtual servers. Other examples of computer systems may include mobile computing devices, such as cellular phones and personal digital assistants, and network equipment, such as load balancers, routers and switches. Additionally, aspects in accord with the present disclosure may be located on a single computer system or may be distributed among a plurality of computer systems connected to one or more communication networks.

For example, various aspects and functions may be distributed among one or more computer systems configured to provide a service to one or more client computers, or to perform an overall task as part of a distributed system. Additionally, aspects may be performed on a client-server or multi-tier system that includes components distributed among one or more server systems that perform various functions. Thus, the disclosure is not limited to executing on any particular system or group of systems. Further, aspects may be implemented in software, hardware or firmware, or any combination thereof. Thus, aspects in accord with the present disclosure may be implemented within methods, acts, systems, system placements and components using a variety of hardware and software configurations, and the disclosure is not limited to any particular distributed architecture, network, or communication protocol. Furthermore, aspects in accord with the present disclosure may be implemented as specially-programmed hardware and/or software.

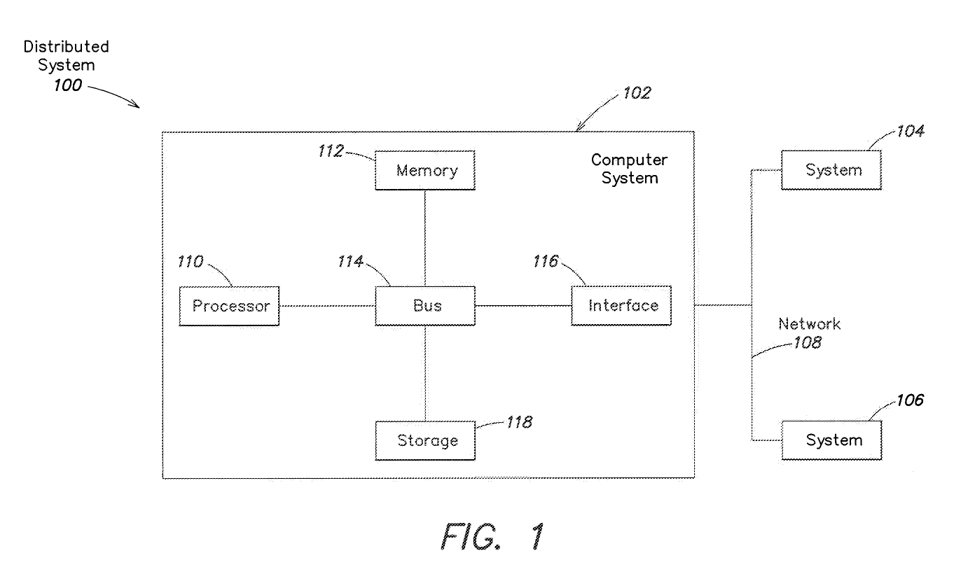

FIG. 1 shows a block diagram of a distributed computer system 100, in which various aspects and functions in accord with the present disclosure may be practiced. The distributed computer system 100 may include one more computer systems. For example, as illustrated, the distributed computer system 100 includes three computer systems 102, 104 and 106. As shown, the computer systems 102, 104 and 106 are interconnected by, and may exchange data through, a communication network 108. The network 108 may include any communication network through which computer systems may exchange data. To exchange data via the network 108, the computer systems 102, 104 and 106 and the network 108 may use various methods, protocols and standards including, among others, token ring, Ethernet, Wireless Ethernet, Bluetooth, TCP/IP, UDP, HTTP, FTP, SNMP, SMS, MMS, SS7, JSON, XML, REST, SOAP, CORBA IIOP, RMI, DCOM, and Web Services. To ensure data transfer is secure, the computer systems 102, 104 and 106 may transmit data via the network 108 using a variety of security measures including TSL, SSL, or VPN, among other security techniques. While the distributed computer system 100 illustrates three networked computer systems, the distributed computer system 100 may include any number of computer systems, networked using any medium and communication protocol.

Various aspects and functions in accord with the present disclosure may be implemented as specialized hardware or software executing in one or more computer systems including the computer system 102 shown in FIG. 1. As depicted, the computer system 102 includes a processor 110, a memory 112, a bus 114, an interface 116 and a storage system 118. The processor 110, which may include one or more microprocessors or other types of controllers, can perform a series of instructions that manipulate data. The processor 110 may be a well-known, commercially available processor such as an Intel Pentium, Intel Atom, ARM Processor, Motorola PowerPC, SGI MIPS, Sun UltraSPARC, or Hewlett-Packard PA-RISC processor, or may be any other type of processor or controller as many other processors and controllers are available. The processor 110 may be a mobile device or smart phone processor, such as an ARM Cortex processor, a Quaicomm Snapdragon processor, or an Apple processor. As shown, the processor 110 is connected to other system placements, including a memory 112, by the bus 114.

The memory 112 may be used for storing programs and data during operation of the computer system 102. Thus, the memory 112 may be a relatively high performance, volatile, random access memory such as a dynamic random access memory (DRAM) or static memory (SRAM). However, the memory 112 may include any device for storing data, such as a disk drive or other non-volatile storage device, such as flash memory or phase-change memory (PCM). Various embodiments in accord with the present disclosure can organize the memory 112 into particularized and, in some cases, unique structures to perform the aspects and functions disclosed herein.

Components of the computer system 102 may be coupled by an interconnection element such as the bus 114. The bus 114 may include one or more physical busses (for example, busses between components that are integrated within a same machine), and may include any communication coupling between system placements including specialized or standard computing bus technologies such as IDE, SCSI, PCI and InfiniBand. Thus, the bus 114 enables communications (for example, data and instructions) to be exchanged between system components of the computer system 102.

Computer system 102 also includes one or more interface devices 116 such as input devices, output devices, and combination input/output devices. The interface devices 116 may receive input, provide output, or both. For example, output devices may render information for external presentation. Input devices may accept information from external sources. Examples of interface devices include, among others, keyboards, mouse devices, trackballs, microphones, touch screens, printing devices, display screens, speakers, network interface cards, etc. The interface devices 116 allow the computer system 102 to exchange information and communicate with external entities, such as users and other systems.

Storage system 118 may include a computer-readable and computer-writeable nonvolatile storage medium in which instructions are stored that define a program to be executed by the processor. The storage system 118 also may include information that is recorded, on or in, the medium, and this information may be processed by the program. More specifically, the information may be stored in one or more data structures specifically configured to conserve storage space or increase data exchange performance. The instructions may be persistently stored as encoded signals, and the instructions may cause a processor to perform any of the functions described herein. A medium that can be used with various embodiments may include, for example, optical disk, magnetic disk, or flash memory, among others. In operation, the processor 110 or some other controller may cause data to be read from the nonvolatile recording medium into another memory, such as the memory 112, that allows for faster access to the information by the processor 110 than does the storage medium included in the storage system 118. The memory may be located in the storage system 118 or in the memory 112. The processor 110 may manipulate the data within the memory 112, and then copy the data to the medium associated with the storage system 118 after processing is completed. A variety of components may manage data movement between the medium and the memory 112, and the disclosure is not limited thereto.

Further, the disclosure is not limited to a particular memory system or storage system. Although the computer system 102 is shown by way of example as one type of computer system upon which various aspects and functions in accord with the present disclosure may be practiced, aspects of the disclosure are not limited to being implemented on the computer system, shown in FIG. 1. Various aspects and functions in accord with the present disclosure may be practiced on one or more computers having different architectures or components than that shown in FIG. 1. For instance, the computer system 102 may include specially-programmed, special-purpose hardware, such as for example, an application-specific integrated circuit (ASIC) tailored to perform a particular operation disclosed herein. Another embodiment may perform the same function using several general-purpose computing devices running MAC OS System X with Motorola PowerPC processors and several specialized computing devices running proprietary hardware and operating systems.

The computer system 102 may include an operating system that manages at least a portion of the hardware placements included in computer system 102. A processor or controller, such as processor 110, may execute an operating system which may be, among others, a Windows-based operating system (for example, Windows NT, Windows 2000/ME, Windows XP, Windows 7, or Windows Vista) available from the Microsoft Corporation, a MAC OS System X operating system available from Apple Computer, one of many Linux-based operating system distributions (for example, the Enterprise Linux operating system available from Red Hat Inc.), a Solaris operating system available from Sun Microsystems, or a UNLX operating systems available from various sources. The operating system may be a mobile device or smart phone operating system, such as Windows Mobile, Android, or iOS. Many other operating systems may be used, and embodiments are not limited to any particular operating system. The computer system 102 may include a virtualization feature that hosts the operating system inside a virtual machine (e.g., a simulated physical machine). Various components of a system architecture could reside on individual instances of operating systems inside separate “virtual machines”, thus running somewhat insulated from each other.

The processor and operating system together define a computing platform for which application programs in high-level programming languages may be written. These component applications may be executable, intermediate (for example, C# or JAVA bytecode) or interpreted code which communicate over a communication network (for example, the Internet) using a communication protocol (for example, TCP/IP). Similarly, functions in accord with aspects of the present disclosure may be implemented using an object-oriented programming language, such as SmallTalk, JAVA, C++, Ada, or C# (C-Sharp). Other object-oriented programming languages may also be used. Alternatively, procedural, scripting, or logical programming languages may be used.

Additionally, various functions in accord with aspects of the present disclosure may be implemented in a non-programmed environment (for example, documents created in HTML, XML or other format that, when viewed in a window of a browser program, render aspects of a graphical-user interface or perforin other functions). Further, various embodiments in accord with aspects of the present disclosure may be implemented as programmed or non-programmed placements, or any combination thereof. For example, a web page may be implemented using HTML while a data object called from within the web page may be written in C++. Thus, the disclosure is not limited to a specific programming language and any suitable programming language could also be used.

A computer system included within an embodiment may perform functions outside the scope of the disclosure. For instance, aspects of the system may be implemented using an existing product, such as, for example, the Google search engine, the Yahoo search engine available from Yahoo! of Sunnyvale, Calif., or the Bing search engine available from Microsoft of Seattle Wash. Aspects of the system may be implemented on database management systems such as SQL Server available from Microsoft of Seattle, Wash.; Oracle Database from Oracle of Redwood Shores, Calif.; and MySQL from Sun Microsystems of Santa Clara, Calif.; or integration software such as WebSphere middleware from IBM of Armonk, N.Y. However, a computer system running, for example, SQL Server may be able to support both aspects in accord with the present disclosure and databases for sundry applications not within the scope of the disclosure.

In addition, the method described herein may be incorporated into other hardware and/or software products, such as a web publishing product, a web browser, or an internet marketing or search engine optimization tool.

Example System Architecture

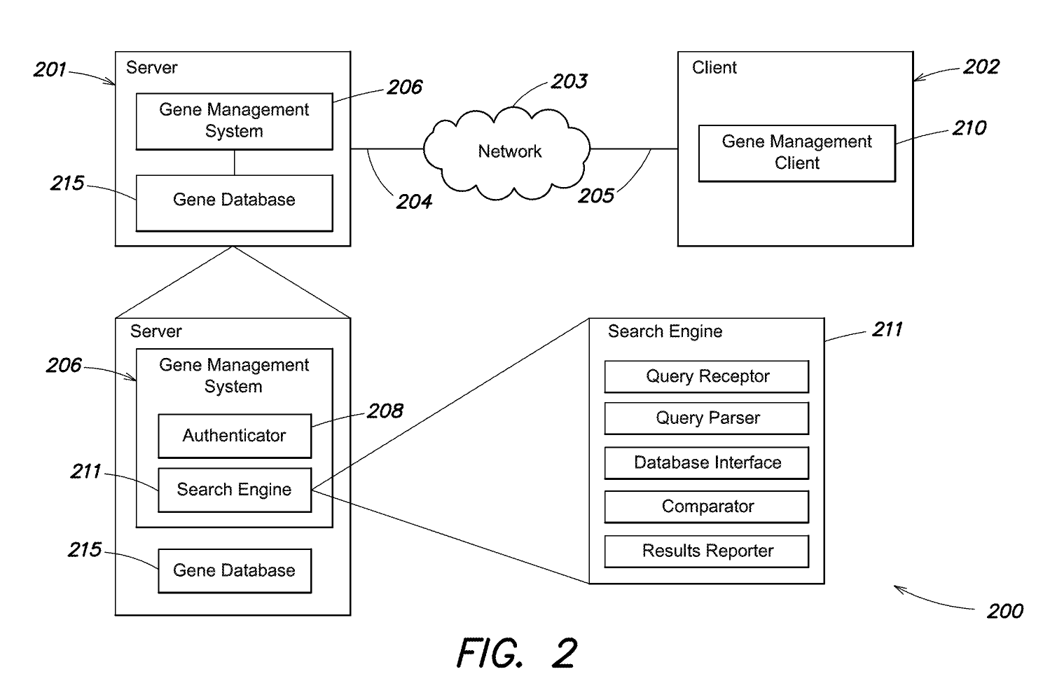

An example system in accordance with aspects of the disclosure can be seen in FIG. 2. The distributed system 200 may include a server system that stores gene data in a relational database library and serves the gene information to clients. In some embodiments, the server includes a process that executes in the memory of the server that serves the gene information. In some embodiments, the server process handles authentication of users, distribution of gene information, and database management functions. The server can also include a component that receives, from one or more client systems, a query that includes, for example, data that relates to a gene sequence or a super-enhancer. The data can include metadata that describes the source of the gene data (e.g., animal, plant, or human identification). Also, the query may include a gene sequence (an entire gene or portion of one) to be identified (e.g., by a client that performs identification) and/or classified as gene sequences of interest according to predetermined criteria.

In some embodiments, the client systems include computer systems communicatively coupled to the server system, such as through a network. In some embodiments, on each client system is loaded a program, subroutine, and/or plug-in executed on the client system and which manages access to gene data stored on the server system. For example, in some embodiments, the program includes a Java applet, when executed, which performs authentication, searching functions on the data stored in the server system database, and displays data to the user. The client system can also include a local database of gene data.

FIG. 2 shows a block diagram of one embodiment of the client-server system. In other embodiments, different architectures can be employed to provide access to gene data, including, for example, a distributed database system accessed by a client (e.g., 202). In further embodiments, the gene data can be distributed throughout client systems and operated as a peer-to-peer gene data network. According to one embodiment, server 201 is, for example, a general purpose computer system specially configured to that perform database storage, searching, and retrieval functions discussed herein. One or more client systems 202 communicate with the server 201 and access gene information. In some embodiments, the server 201 and the client 202 are coupled via one or more communication links such as, for example, a link 204 which couples the server 201 to a network 203, such as the Internet or an intranet, and a link 205 which couples the client 202 to the network 203.

In some embodiments, the server 201 includes a gene management system 206 that performs authentication and database functions with entries of a gene database 215. In some embodiments, the gene management system 206 includes an authenticator 208 that authenticates users, systems, or processes to access entries in the gene database 215. In some embodiments, the gene management system 206 includes a database management system (DBMS) that provides an interface to the gene database 215. The DBMS may be, for example, an Oracle database system and the gene database 215 may be an Oracle relational database. The gene management system 206 may also include a search engine 211 that indexes the gene database 215 and provides searching capabilities to one or more clients 202.

Access to the database may be provided, for example, through one or more clients 202 that execute a client program 210. In some embodiments, the client program 210 is provided by the server 201. The client 202 may be, for example, a general purpose computer system having a processor that executes an operating system and software programs programmed in one or more programming languages. The client 202 may execute a program known as a browser, and the client program 210 may be executed within the browser. The client program 210 may include, for example, a markup language document (e.g., an HTML document) that provides access to functions on the server 201 (e.g., via cgi script or other method). The client program 210 may be, for example, an executable program written in a compiled (e.g., C++) or interpreted programming language (e.g., Java), and viewed in an interface of the browser executed on the client 202. The client program 210 may be provided for accessing information in the gene database 215. In some embodiments, the client program 210 includes an authenticator that authenticates a user to server 201.

The browser program may display a user interface on a client system. A user can access the user interface displayed in the browser program to enter gene search criteria. According to one embodiment, the user interface is configured to accept input (e.g., search criteria such as keywords, gene sequences, etc.) from a user. The search engine 211 may provide searching capabilities, for example, of the gene database 215, which can include gene data obtained from the server 201. In one example, the search engine 211 receives search criteria from the user input and executes search functions on the database to return matching results. The results can include identification of candidate super-enhancers, genes, gene sequence identifiers, information on membership within in a complex, interaction information (e.g., interacts with a member of a complex), chromatin-associated domain information, among other options.

According to one embodiment, the search engine 211 can include modules, such as a query receptor 220, a query parser 221, a database interface 222, a comparator 223, and a result reporter 224. For example, the search engine 211 can receive search queries on the query receptor 220. The query parser 221 can receive the search query from the query receptor 220 and parse the search query into individual keywords and/or gene sequences or other appropriate parsed data components. The database interface 222 can be used to input the search query and to retrieve results from the gene database 215. The comparator 223 can compare the results with the search query or compare the results to each other to determine a relevance of the results. In one example, the comparator 223 evaluates the search query against gene information (e.g., gene information encoded in Table 1) to identify super-enhancers, candidate treatment options, etc. The results can be listed and/or ranked, for example, based on the likelihood that a result is a matching super-enhancer and/or a treatment option. In another example, potential results generated from a search query execution are evaluated against each other to rank the results based on a relative likelihood that a result is a super-enhancer and/or a candidate treatment option. The result reporter 224 can provide the results to an interface. The search engine 211 can also be used to search databases other than the gene database 215. For example, the search engine 211 can be used to search databases including knowledge of the association, correlation, or role of genes and enhancer components to cell states.

In some embodiments, the gene management system 206 performs acts of storing gene information in a database, receiving requests for identifying gene information including search parameters, and returning results of the search. While the distributed system 200 is shown as including the server 201 and the client 202 communicating over the network 203, the components of the server 201 and the client 202 can be included on a single computer or computing system. For example, a user can execute queries on the server 201 directly. In some embodiments, the distributed system 200 can be used to select, evaluate, and classify genes associated with super-enhancers. The distributed system 200 can also be used to select, evaluate, and classify super-enhancer components.

Exemplary Process

Having described various aspects of a system for evaluating genes, the operation of such a system is now described.

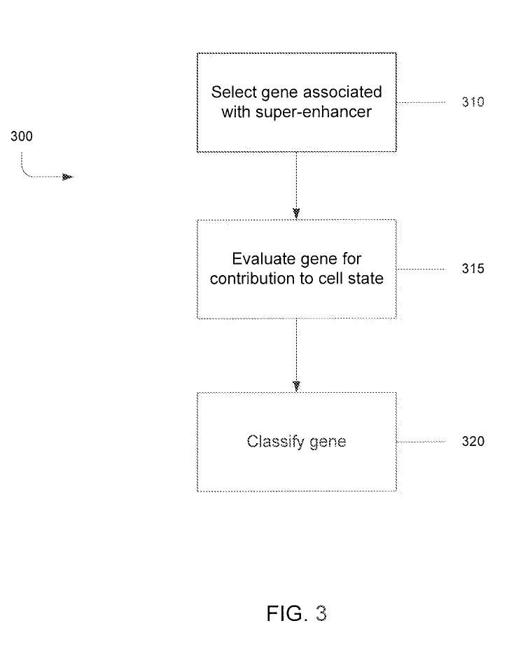

A process according to some embodiments is described with reference to FIG. 3, illustrating an exemplary process 300 of evaluating a target gene. In some embodiments, the process 300 may be embodied in a module, plugin, or component of the distributed system 200 shown in FIG. 2. In other embodiments, the process 300 may be embodied in a stand-alone application that performs the functions described herein.

Process 300 includes selecting a gene associated with a super-enhancer at act 310. The gene can be selected based at least in part on the association with the super-enhancer. In some embodiments, search queries can be executed on the distributed system 200, for example, of the gene database 215, to identify a target gene for selection.

In act 315, a selected gene is evaluated. The evaluation can include acquiring knowledge of the relevance of the selected gene to a cell state. For example, evaluation can include capturing information on an association, correlation, or role of the selected gene to the cell state (e.g., the cell state can include cancer cell). The cell state can include a state of health or disease or disorder and/or a state of differentiation. In some embodiments, search queries can be executed on the distributed system 200, for example, of knowledge databases to acquire the knowledge associated with the target gene. In some examples, the search queries are executed to return information on a gene and any associations with the gene, any correlations, and/or any role known for the target gene which can also be filtered by any cell state associated with the target gene. The returned information can include information on the effect of one or more of the following factors on cell state (e.g., cancer), or on a parameter associated with the cell state (e.g., proliferation, invasiveness, epithelial to mesenchymal transition, likelihood to metastasize, or cell death): a) the effect of modulation (e.g., down modulation or up modulation) of the level of gene expression, or of a gene product on the cell state; b) the effect of gene targeting (e.g., by anti-sense or RNAi) of the selected gene; c) the effect or results of a synthetic lethal screen; d) the effect of a genetic event involving the selected gene (e.g., a copy number alteration, e.g., amplification or deletion, a rearrangement, e.g., a translocation, deletion, insertion, or point mutations, or other mutation); e) expression or hybrid capture sequencing data (e.g., from a source such as the Cancer Cell Line Encyclopedia); the effect of overexpressing a dominant negative mutant of the selected gene; or g) the effect of reducing the copy number of the gene by genome editing or introducing an analog sensitive mutation into the gene by genome editing, among other options.

In act 320, the selected gene is classified based, at least in part, on the evaluation. The classification of the selected gene can include classification as a candidate target gene for the treatment or diagnosis of a subject having the cell state. In some embodiments, the genes can be classified on the distributed system 200, for example, by using metadata tags or some other appropriate designation system.

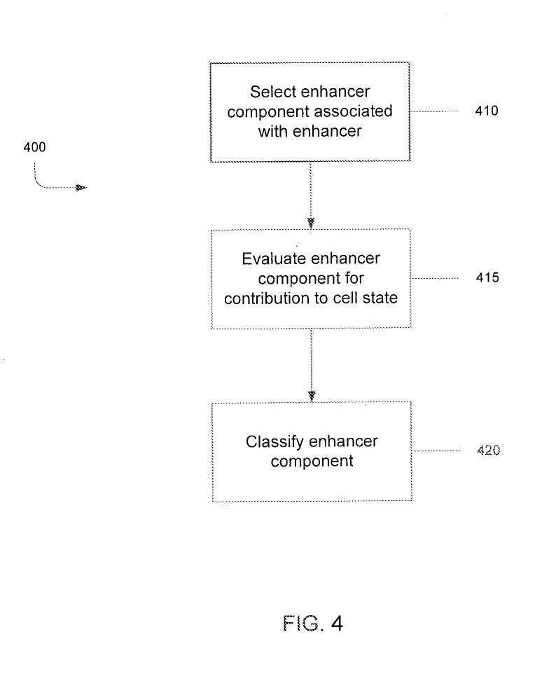

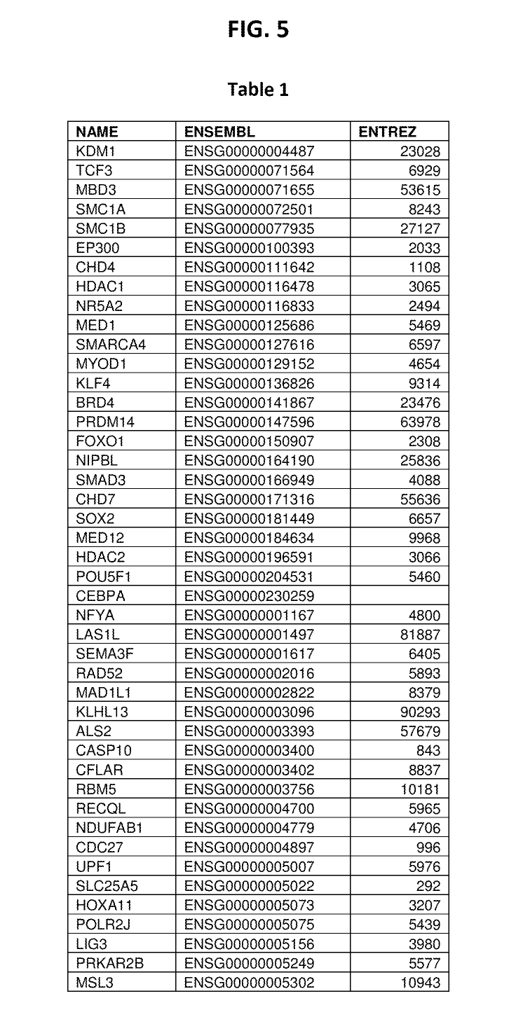

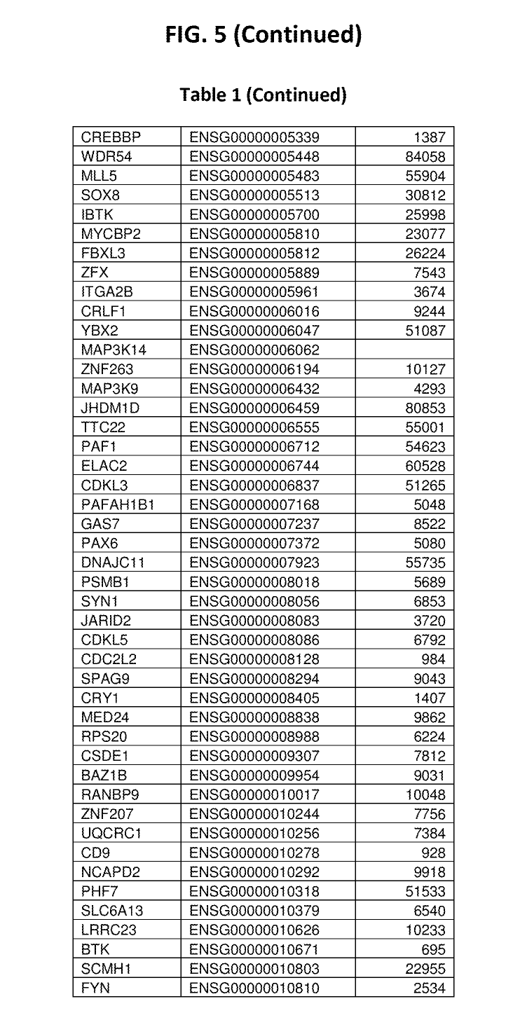

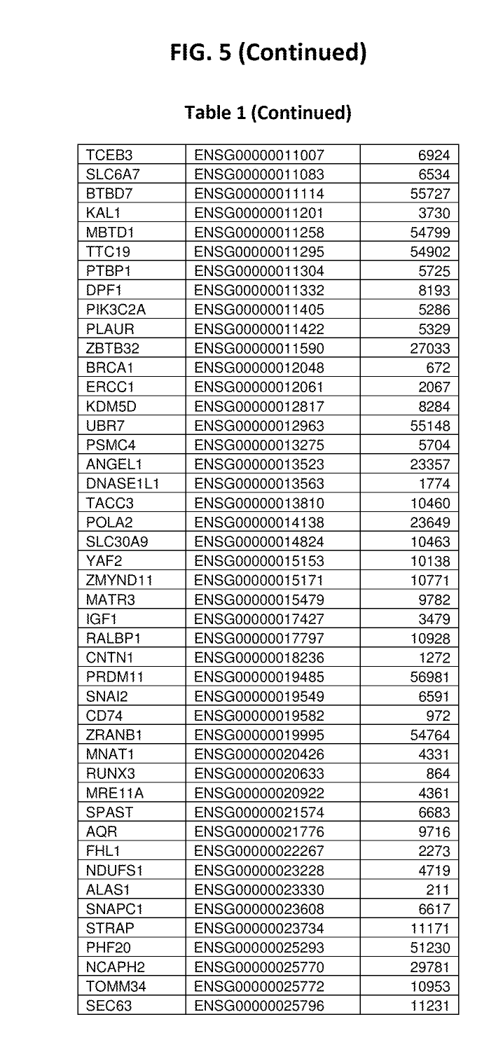

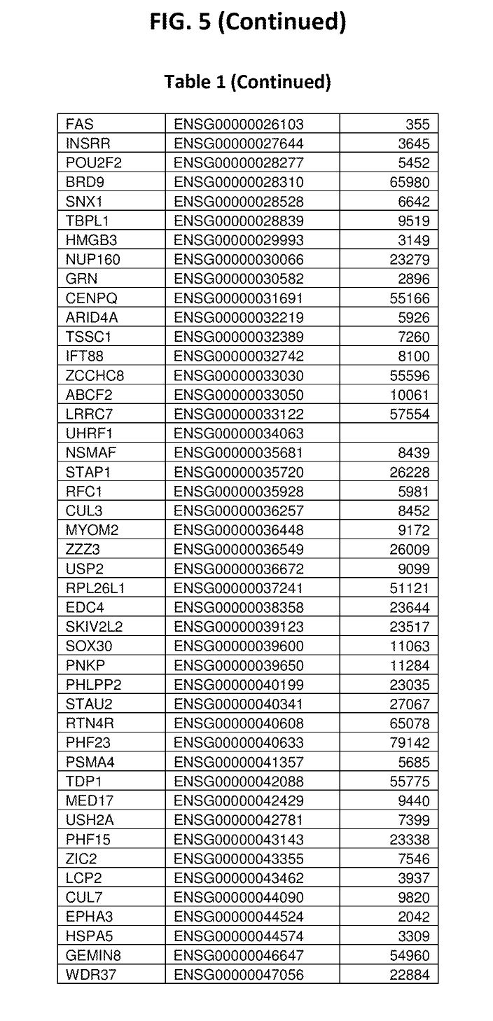

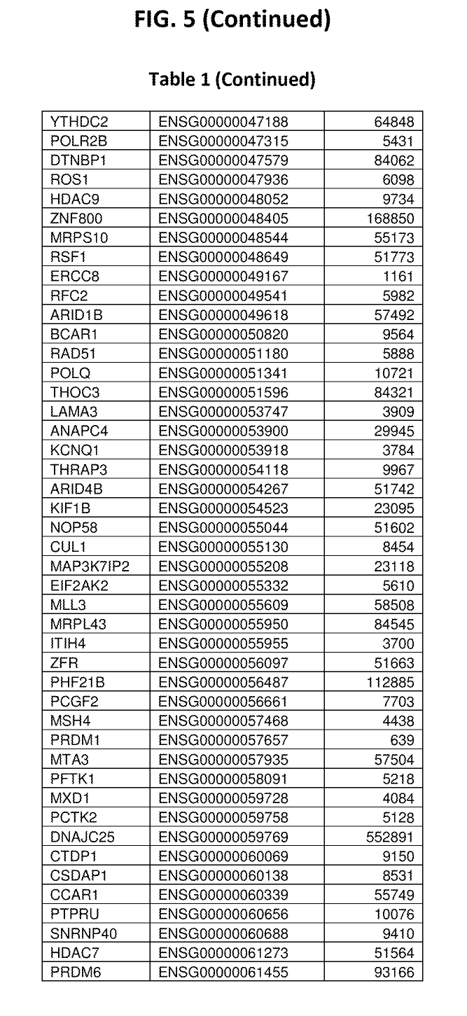

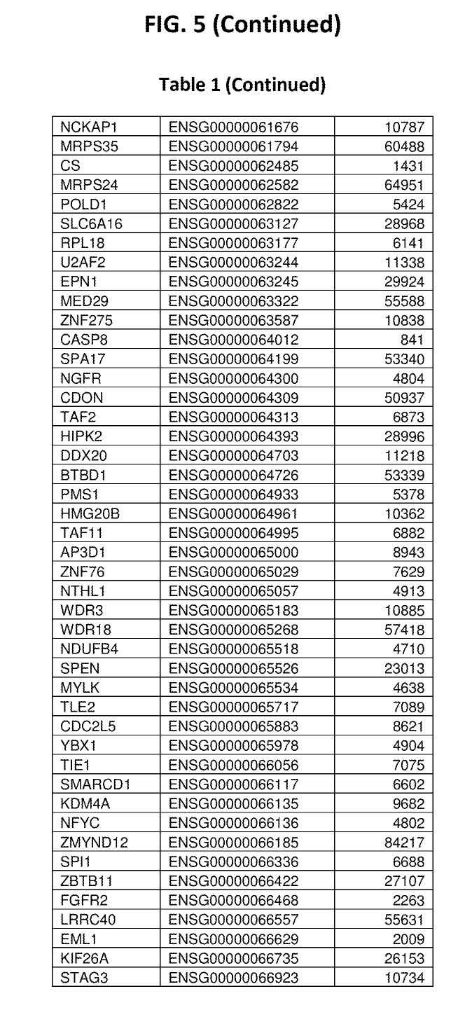

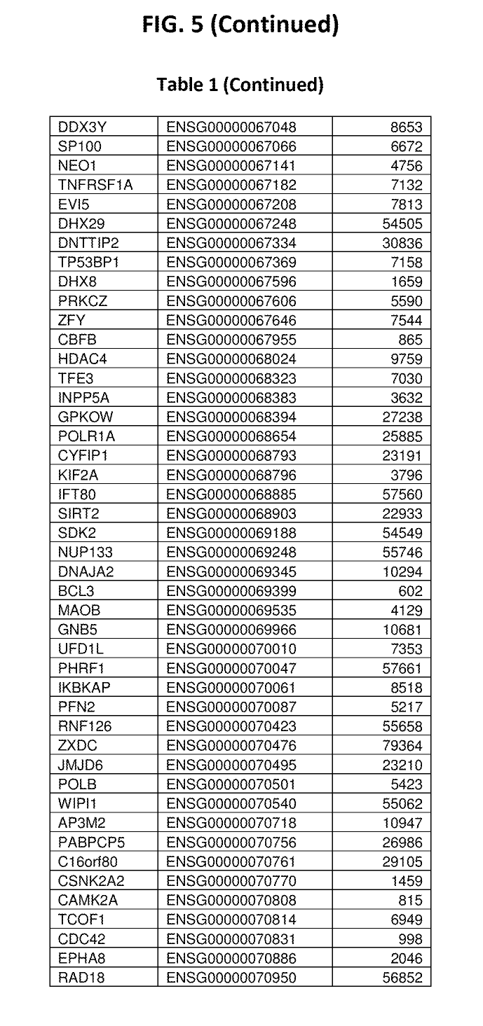

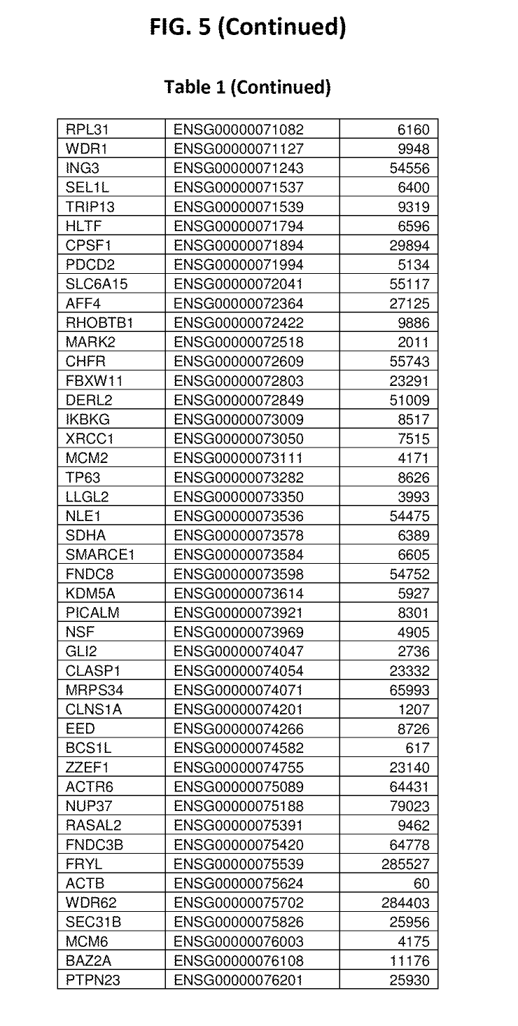

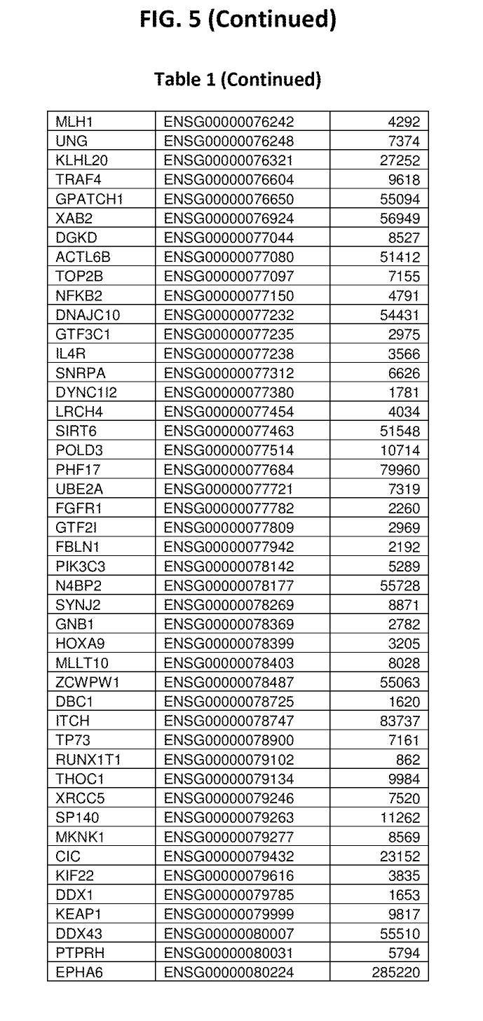

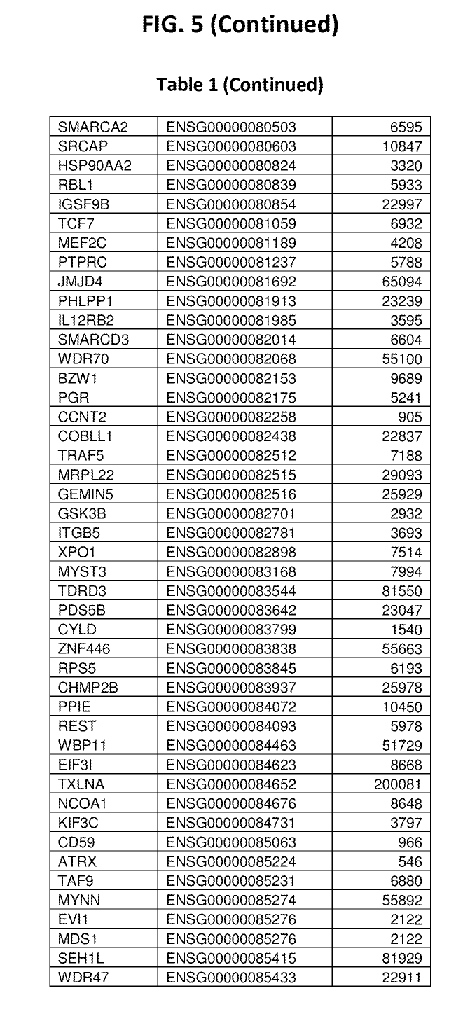

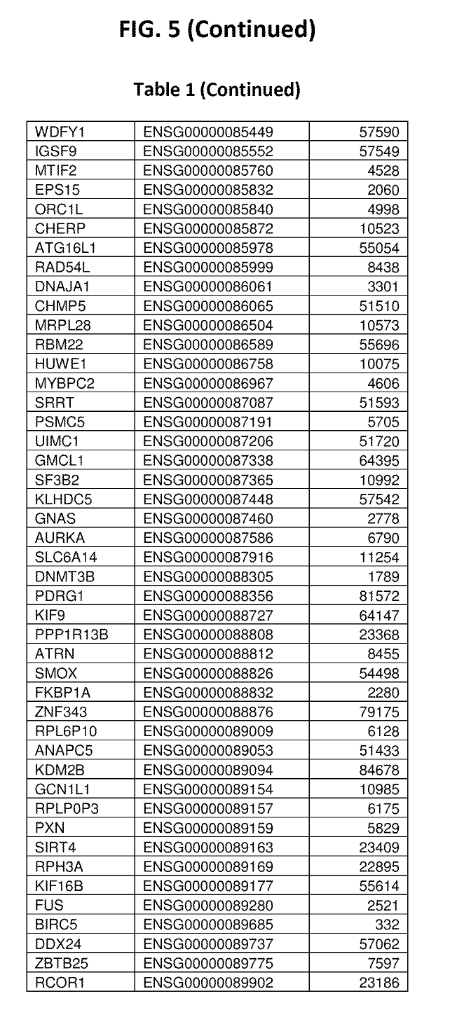

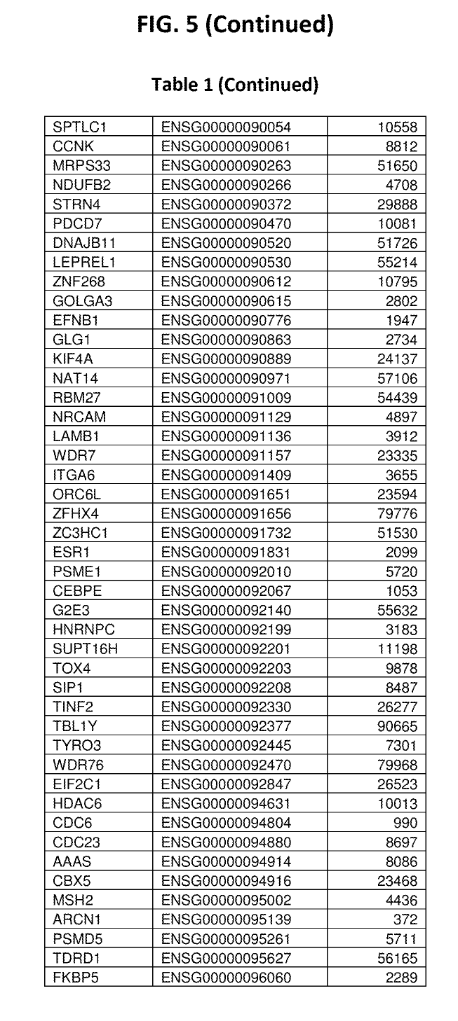

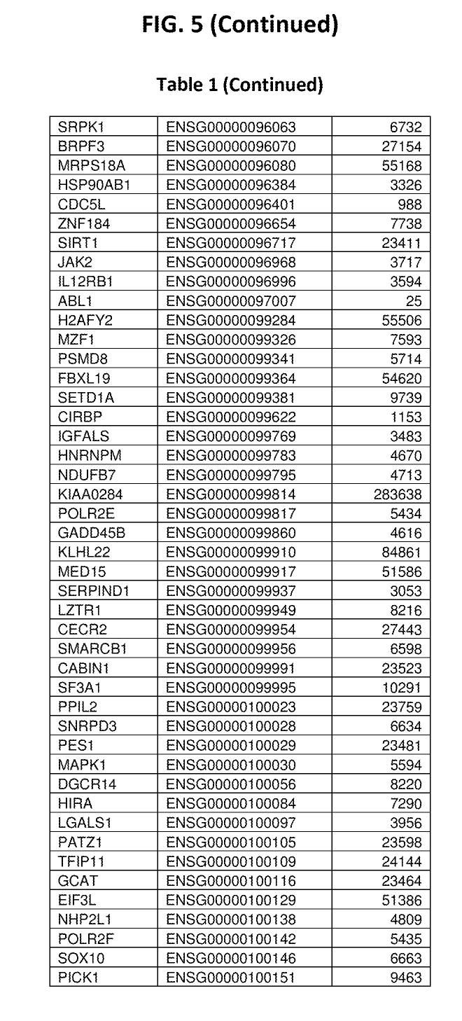

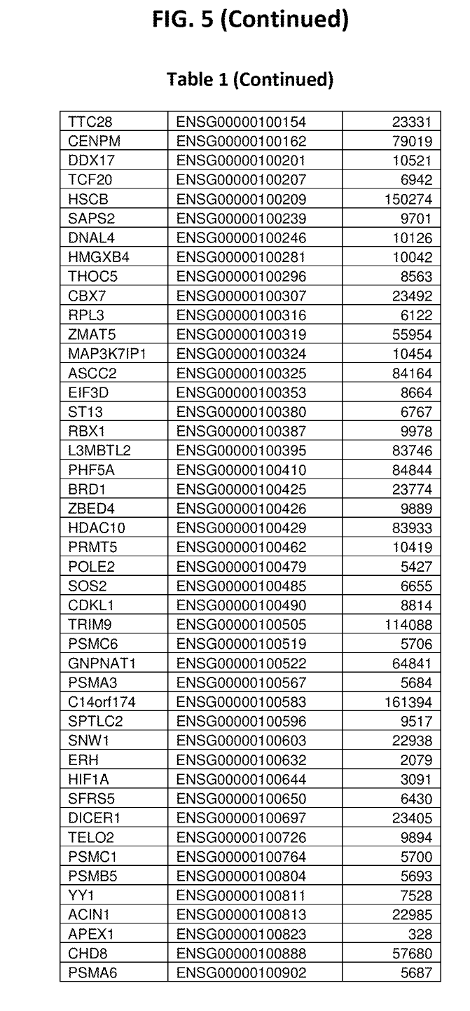

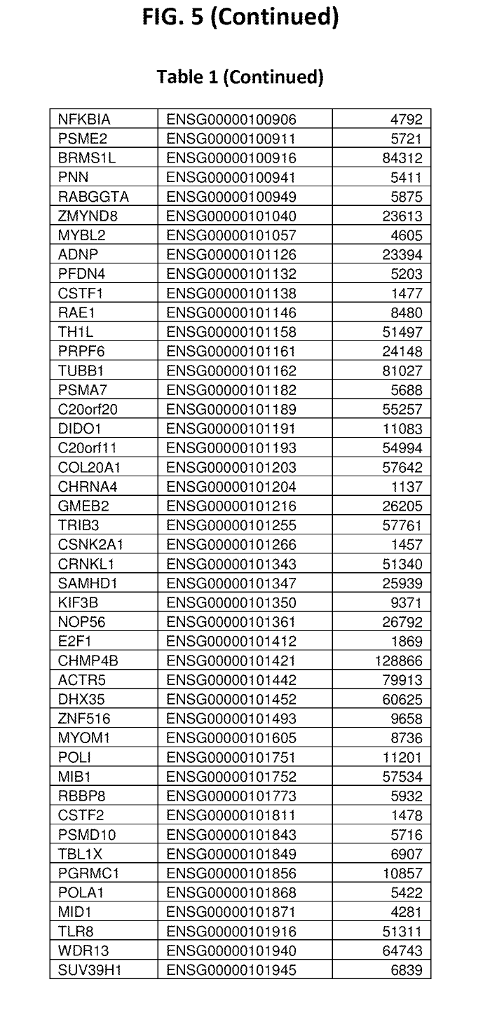

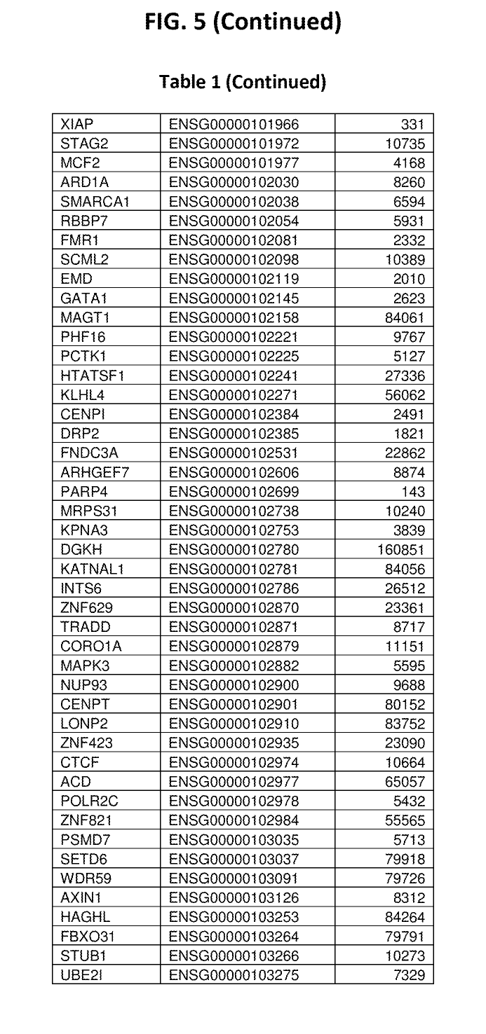

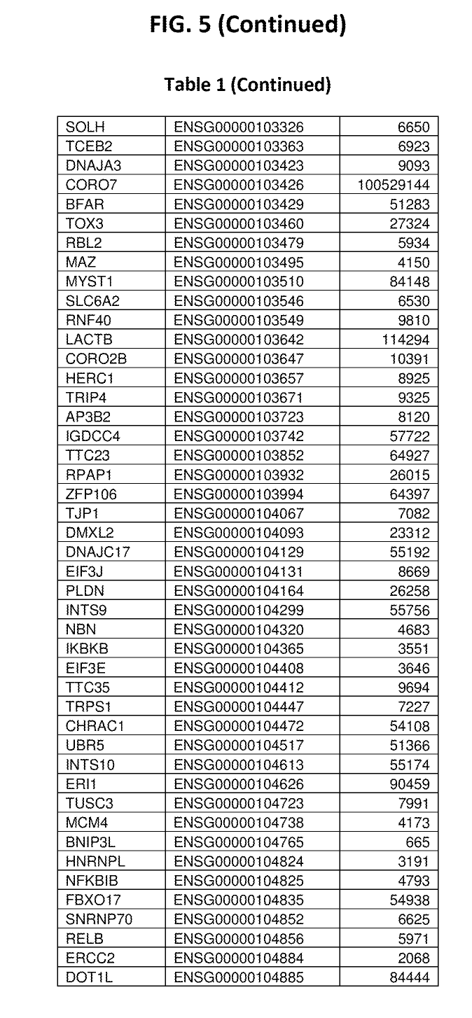

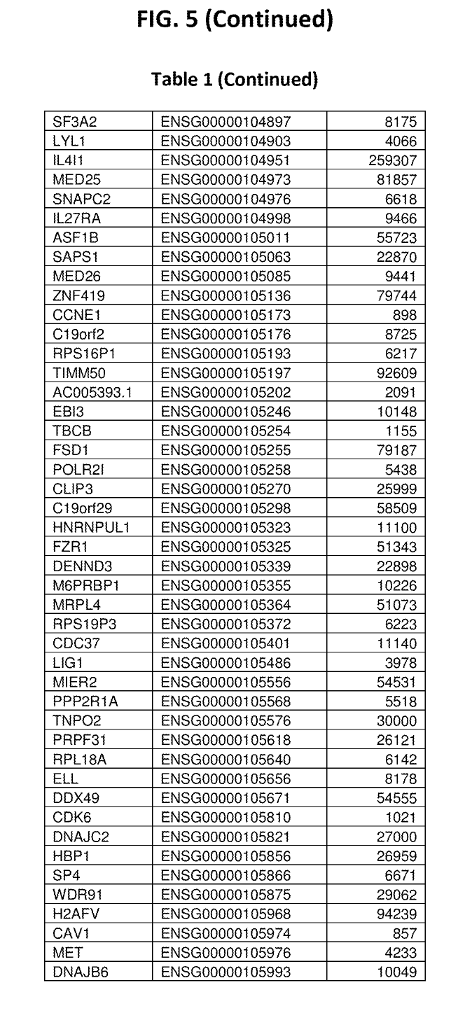

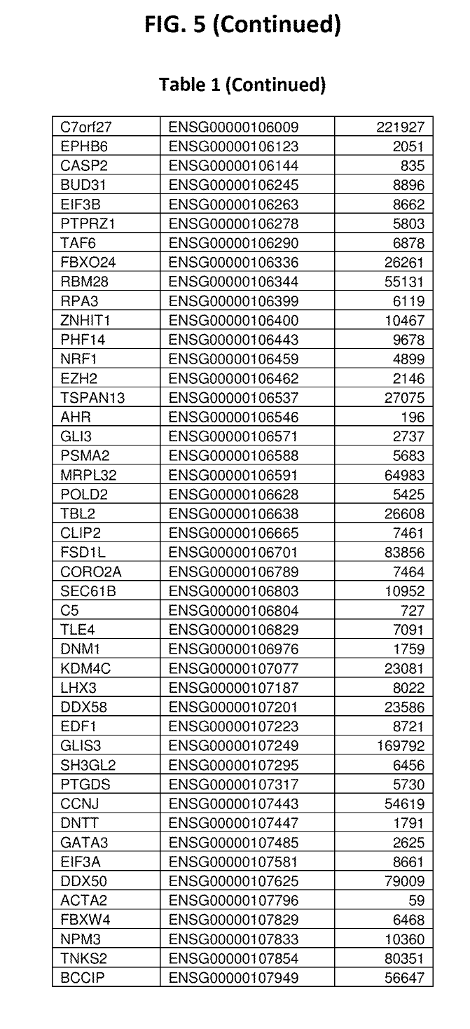

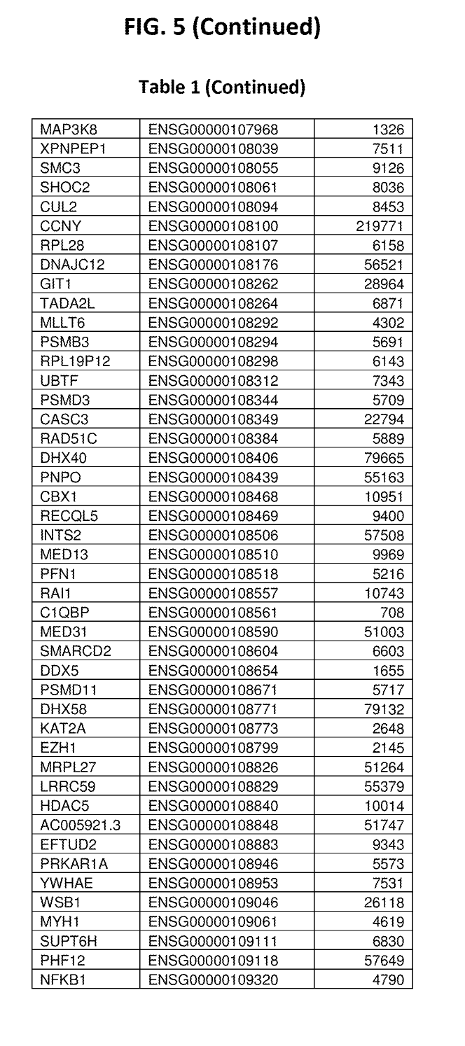

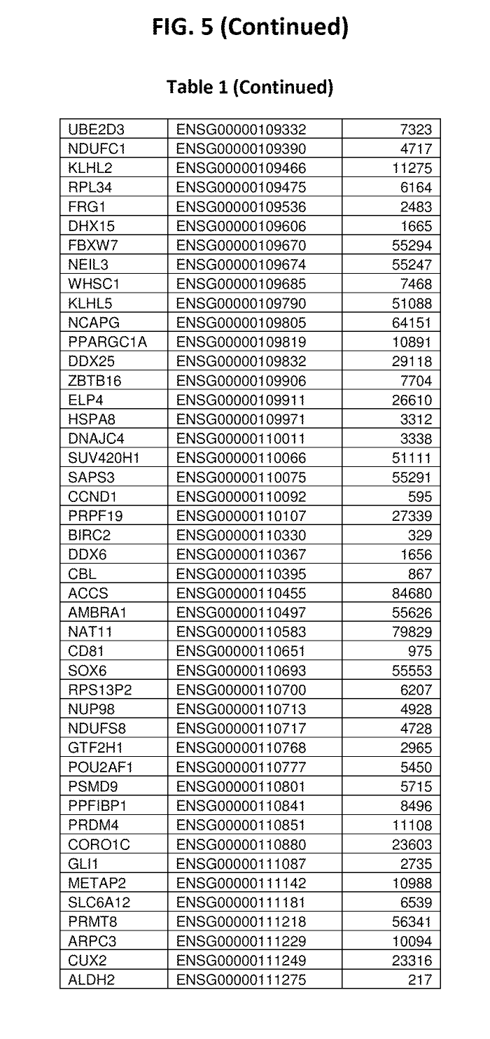

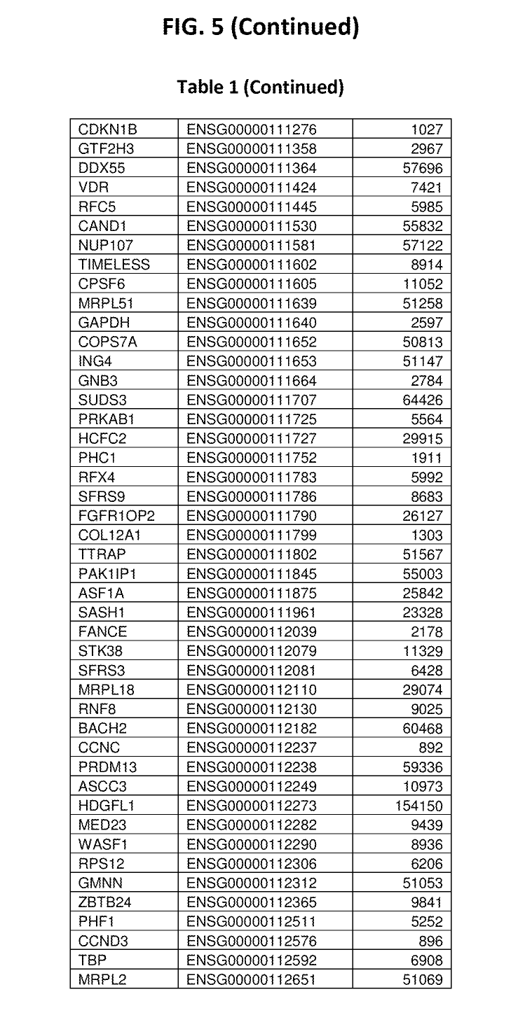

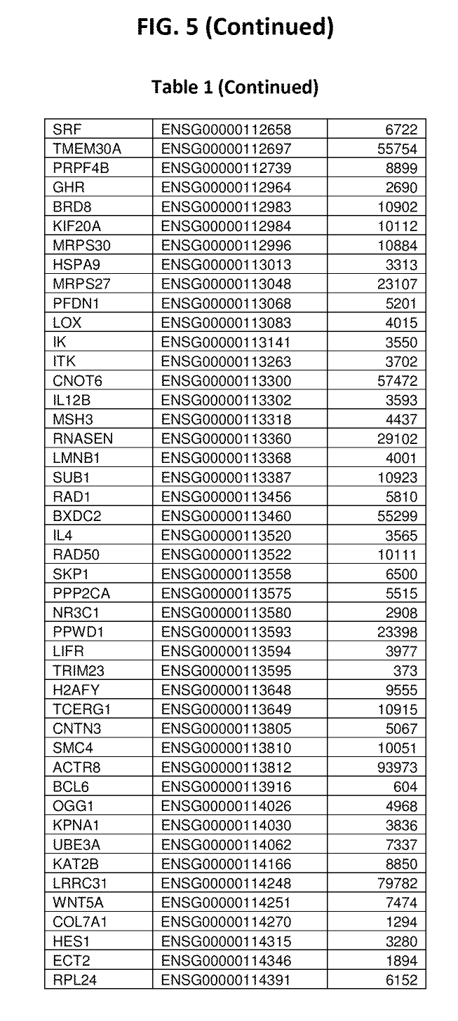

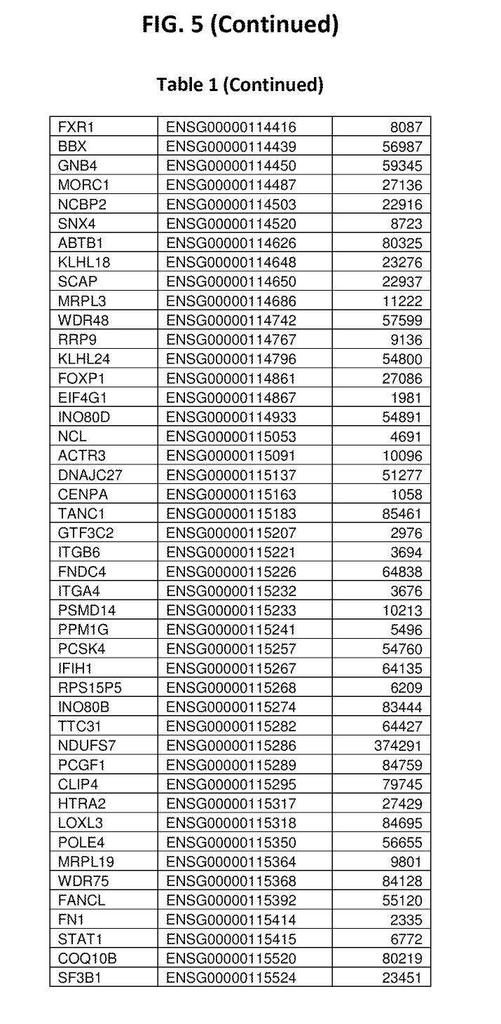

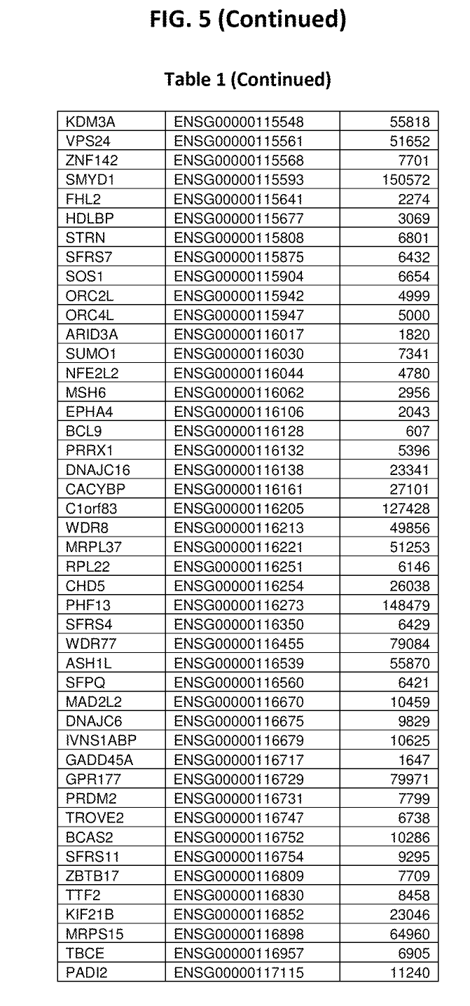

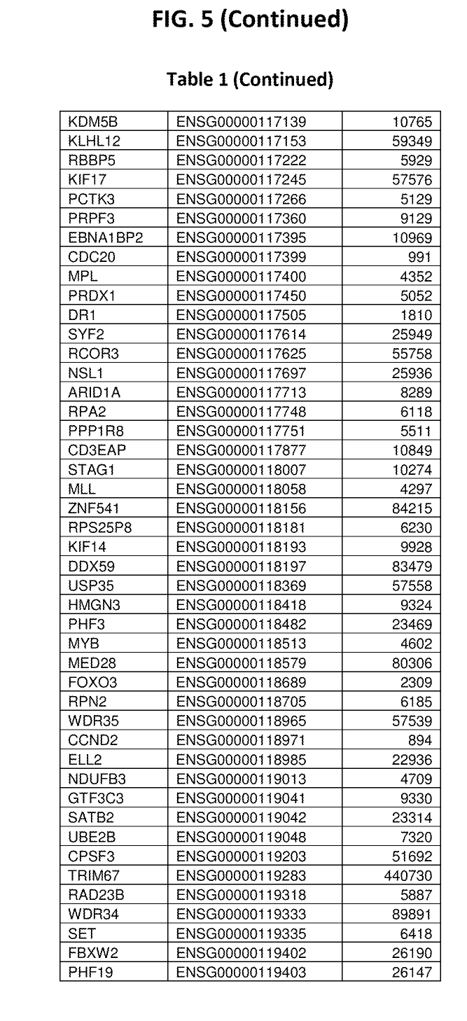

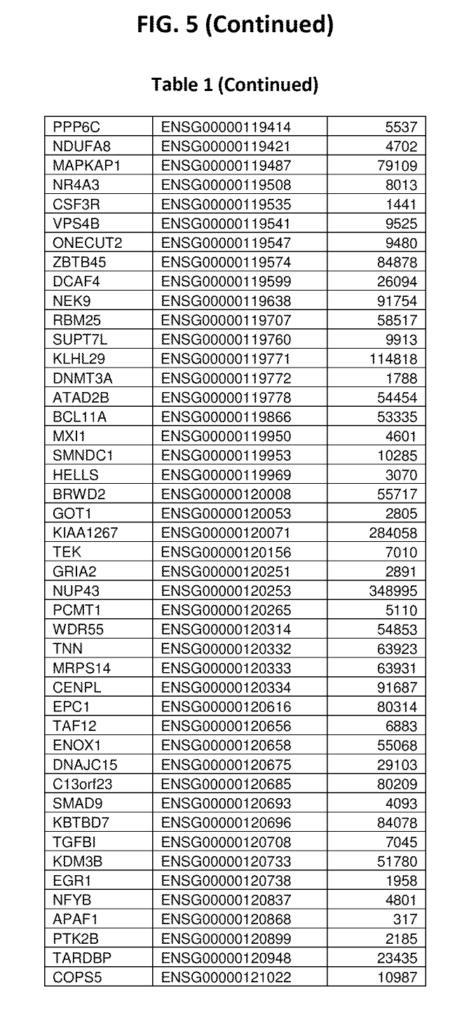

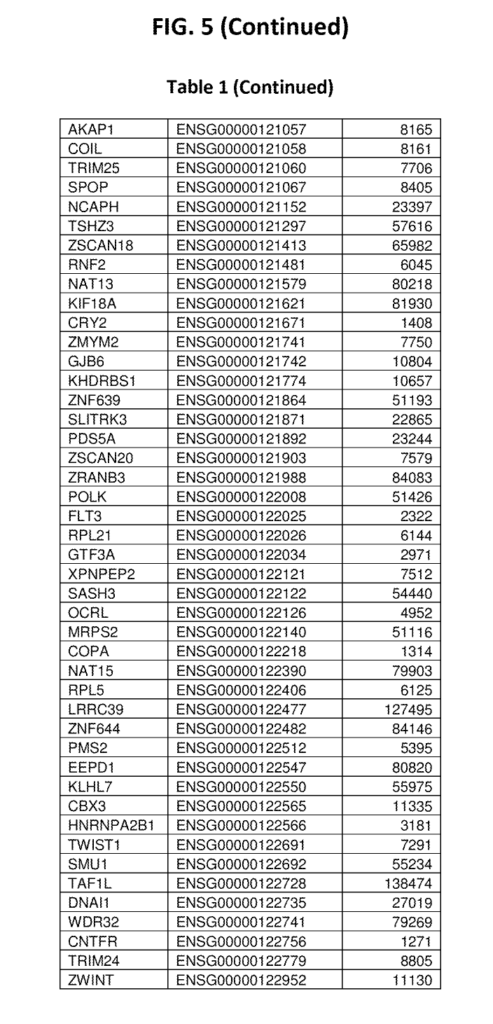

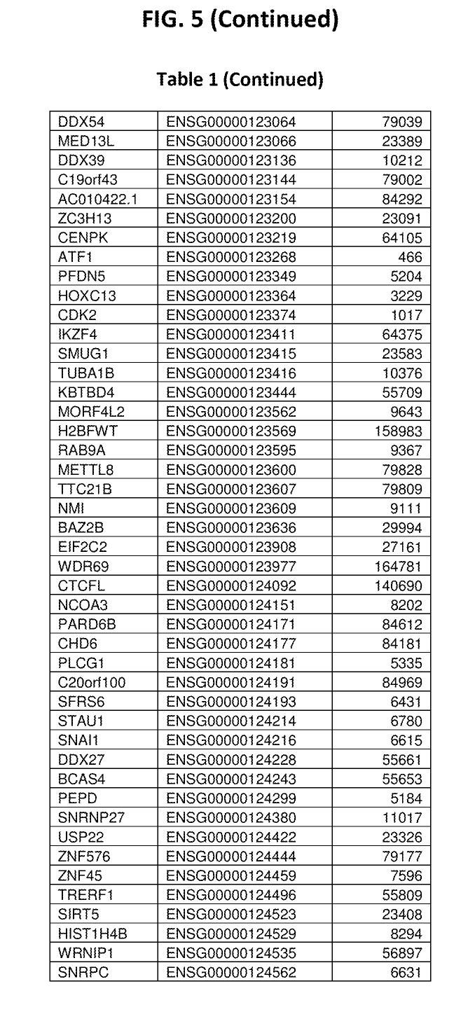

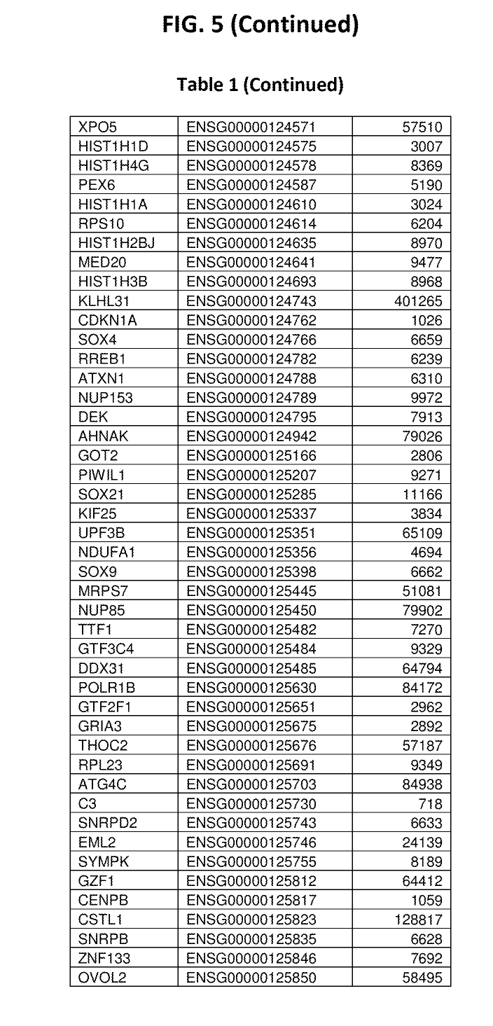

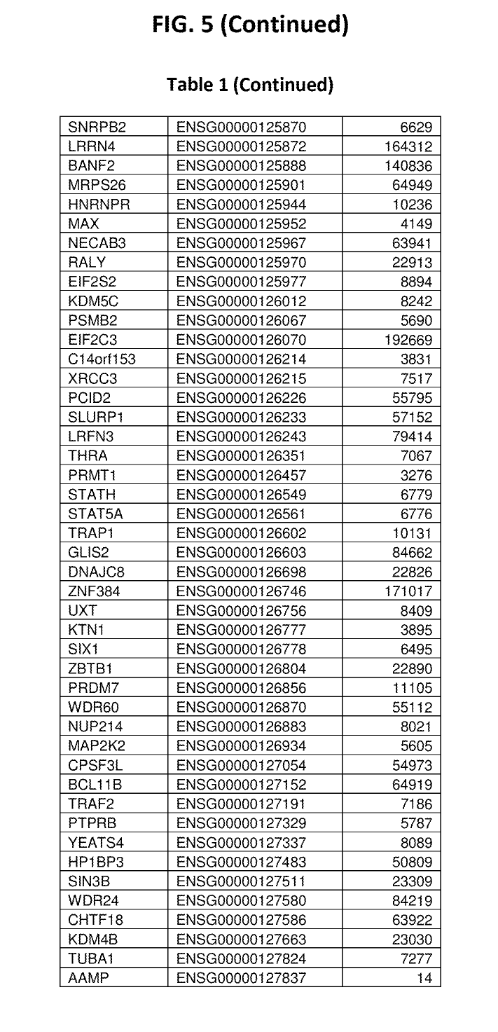

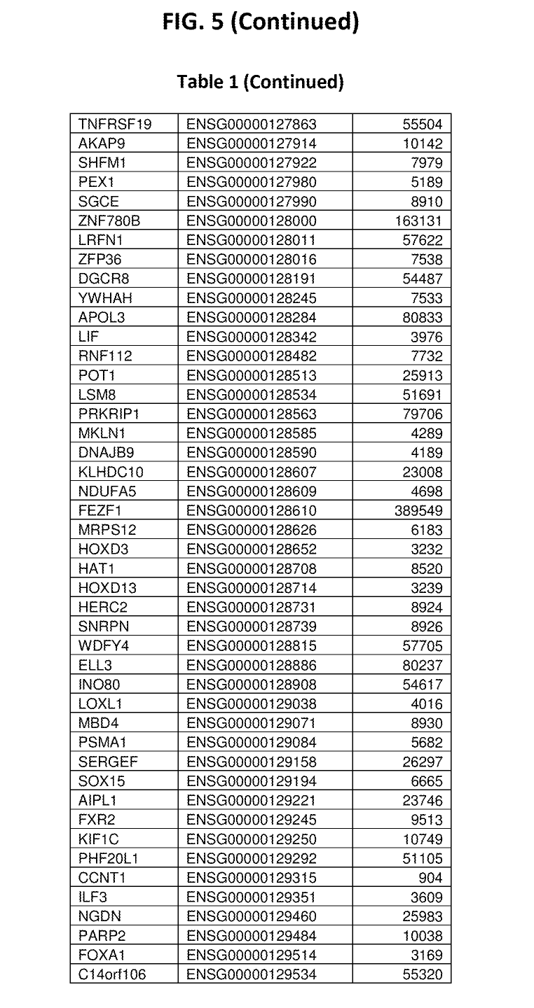

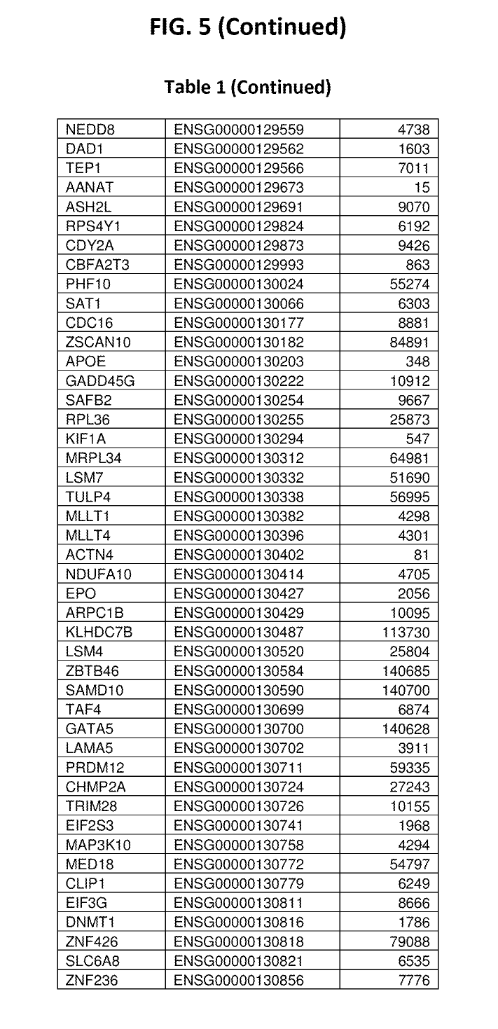

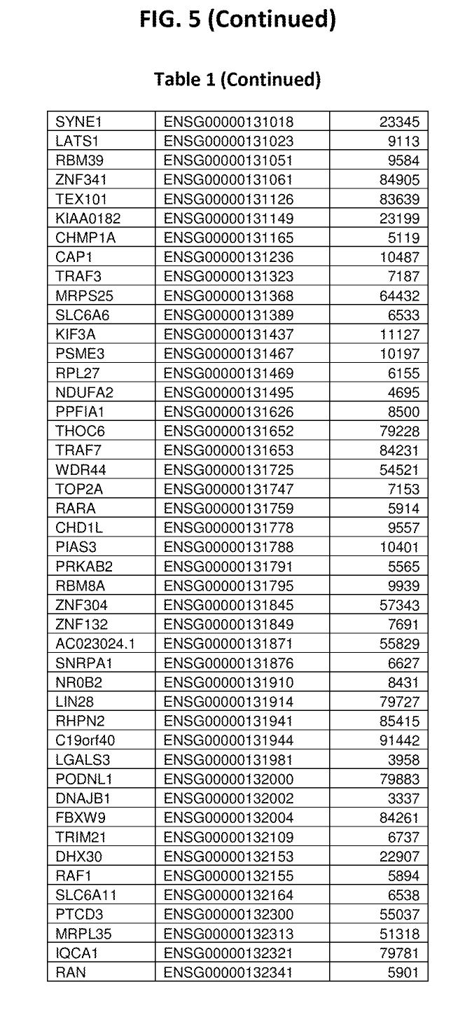

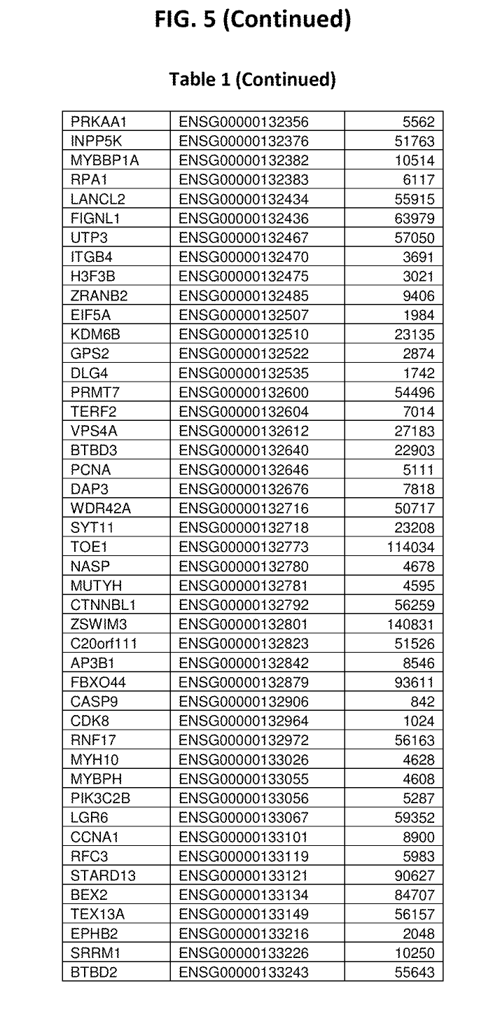

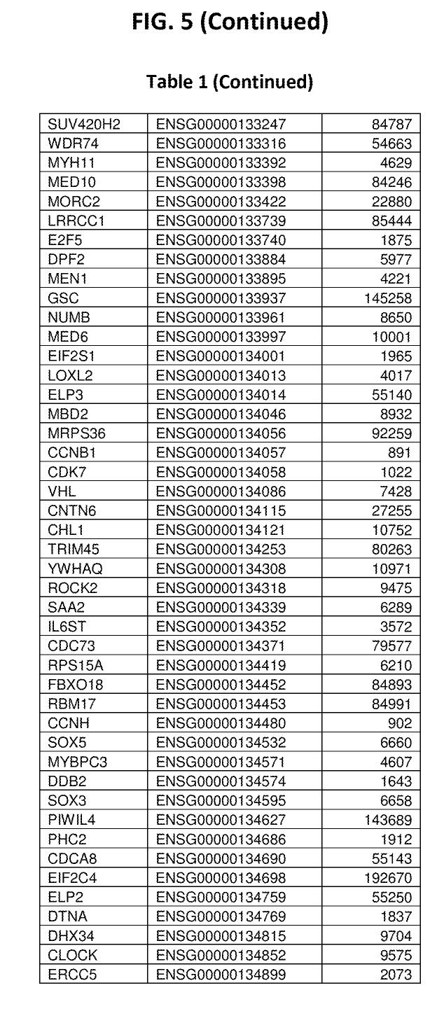

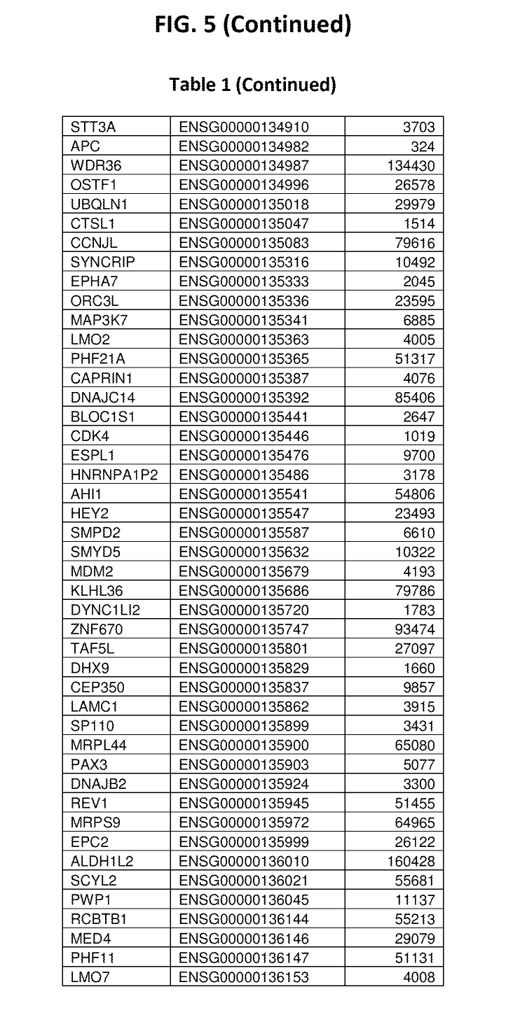

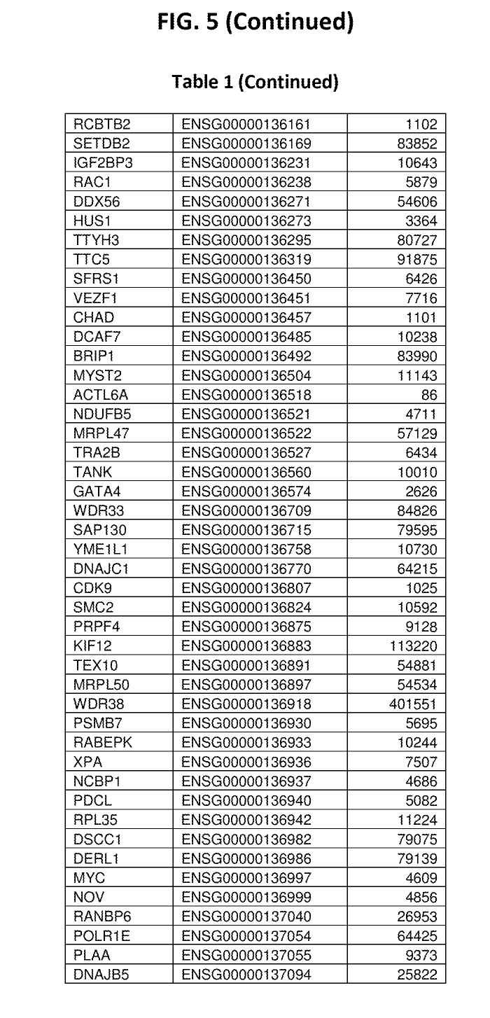

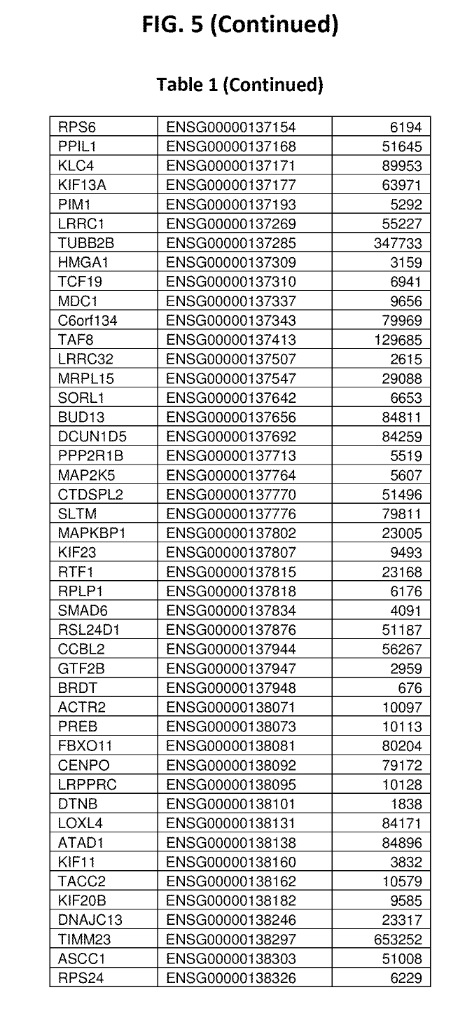

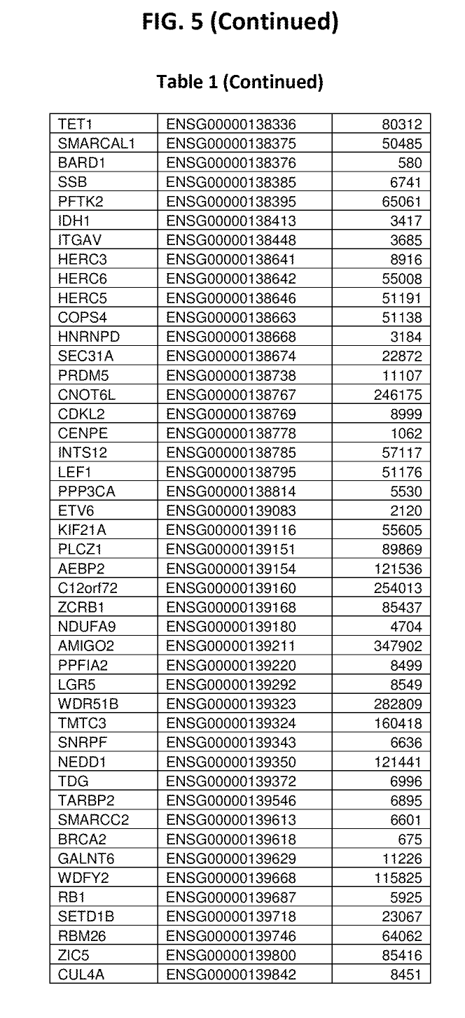

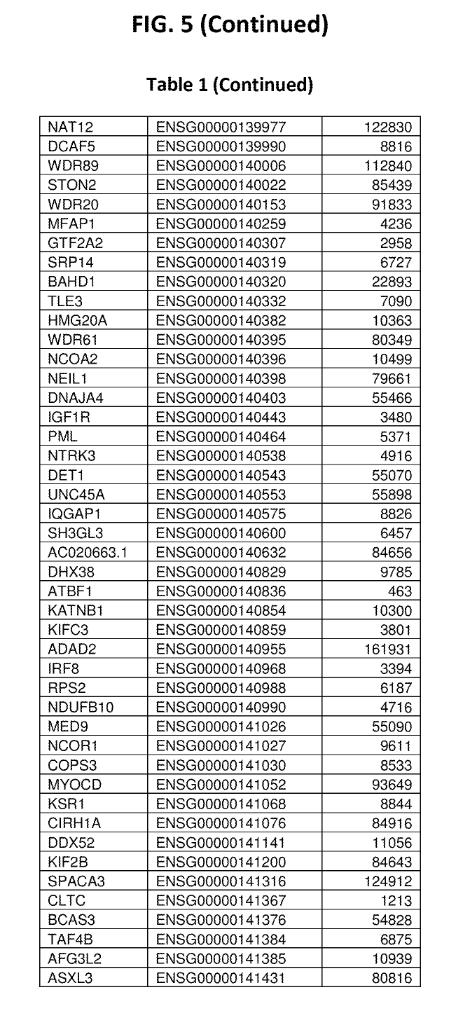

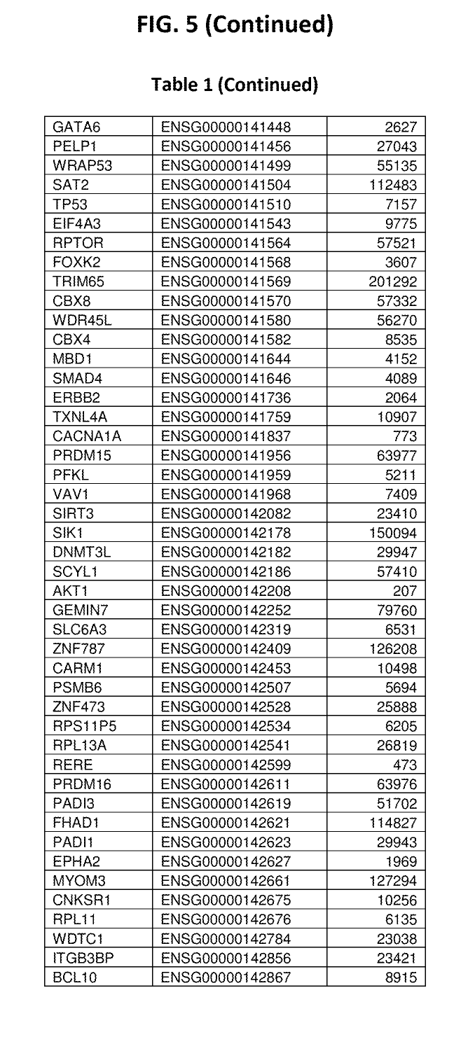

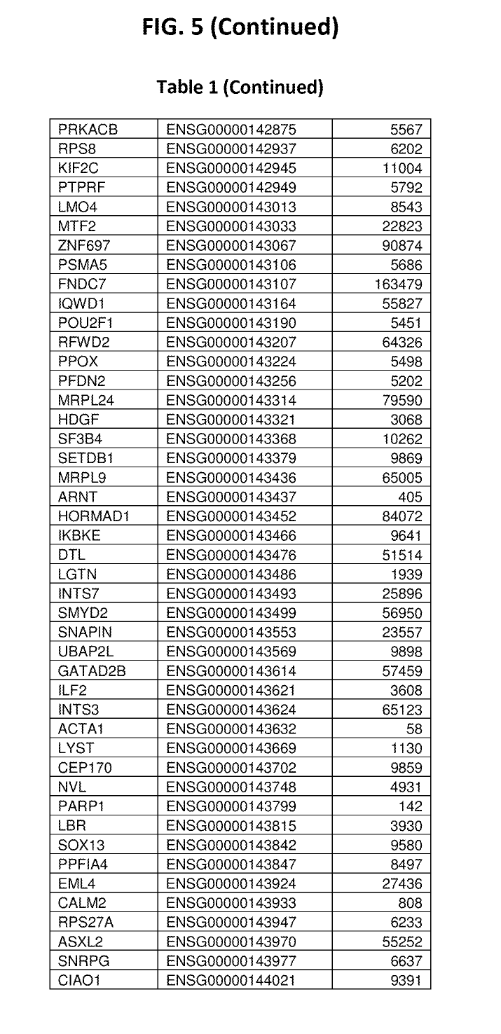

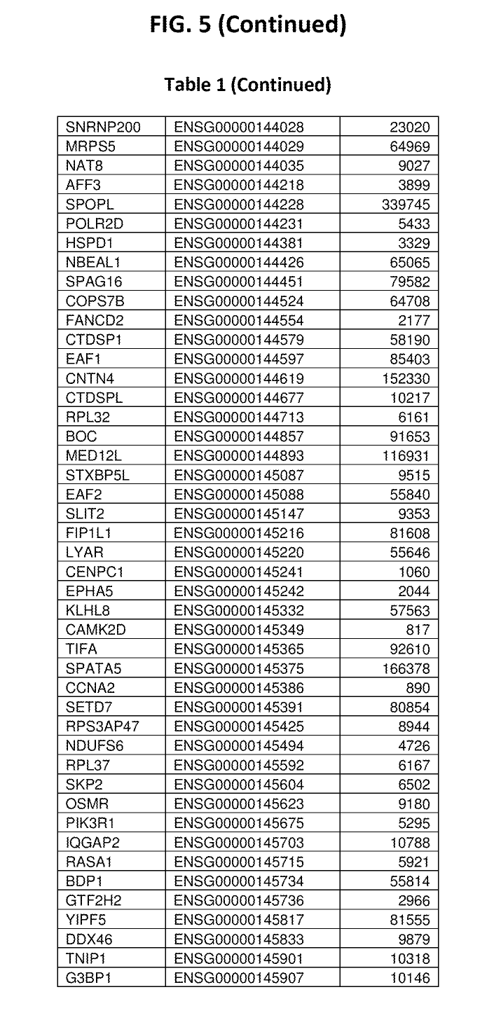

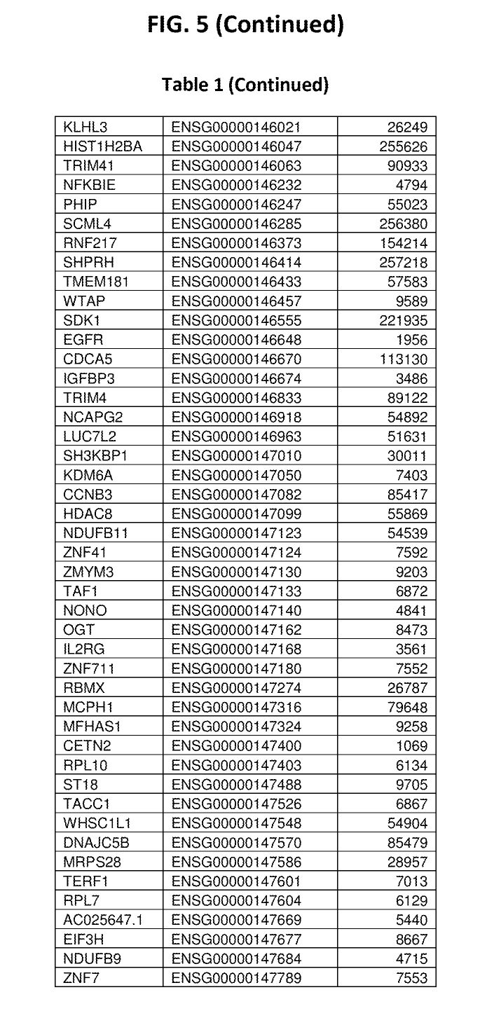

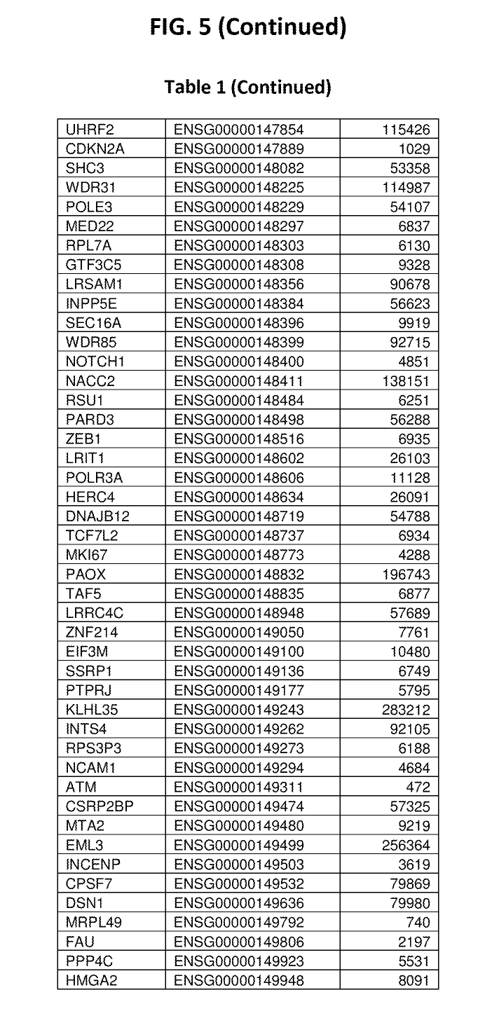

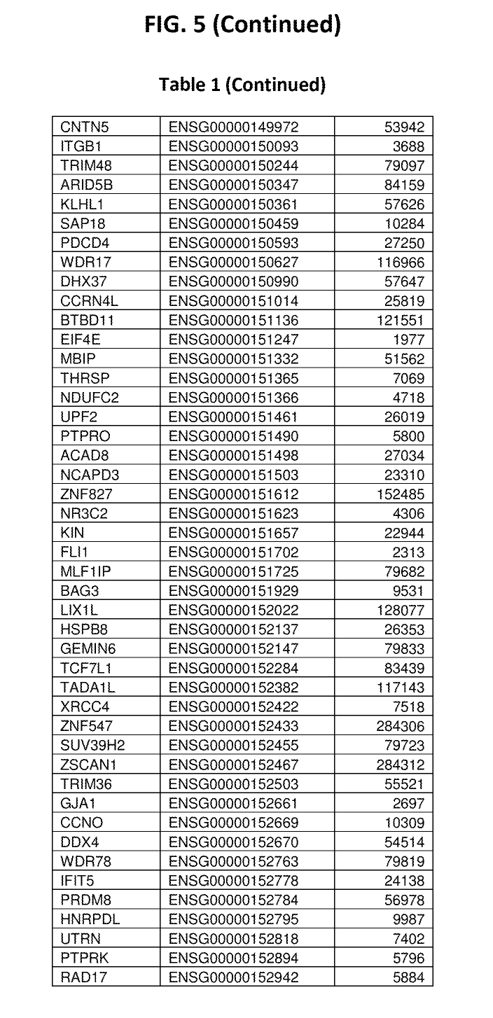

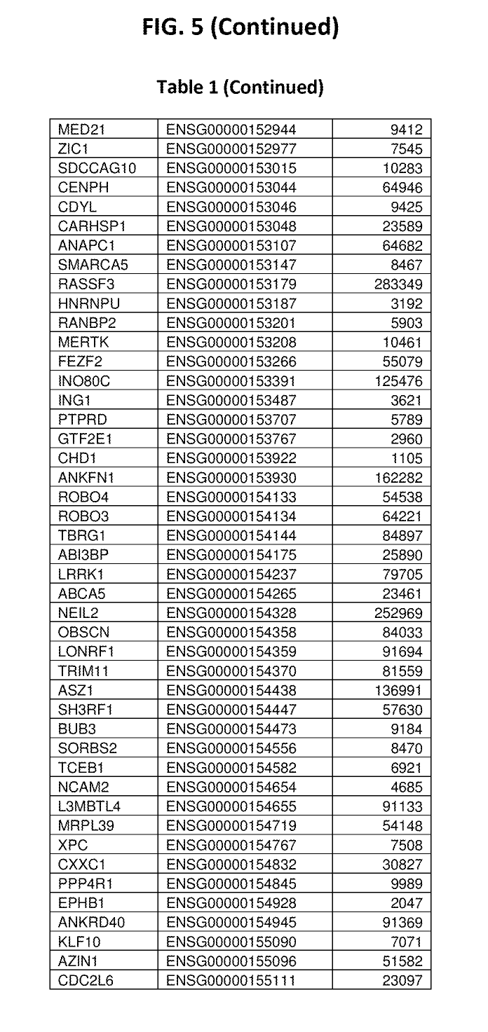

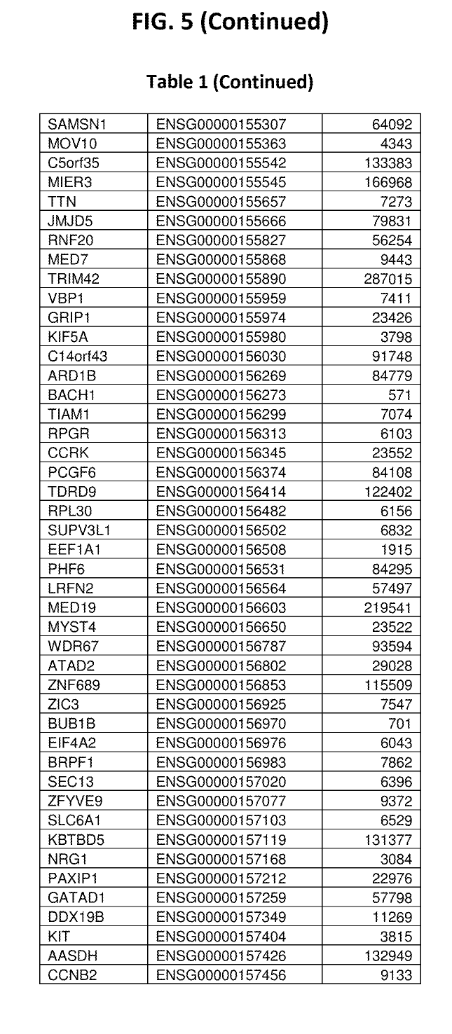

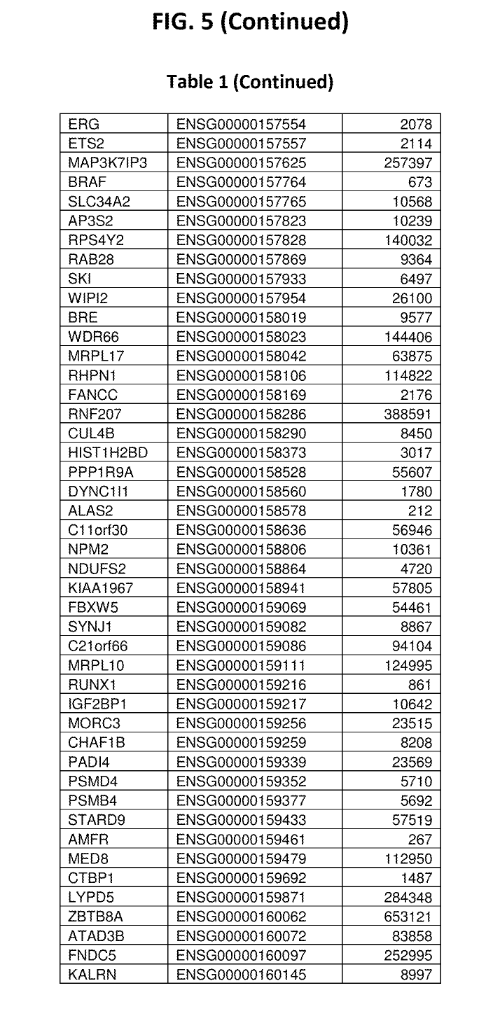

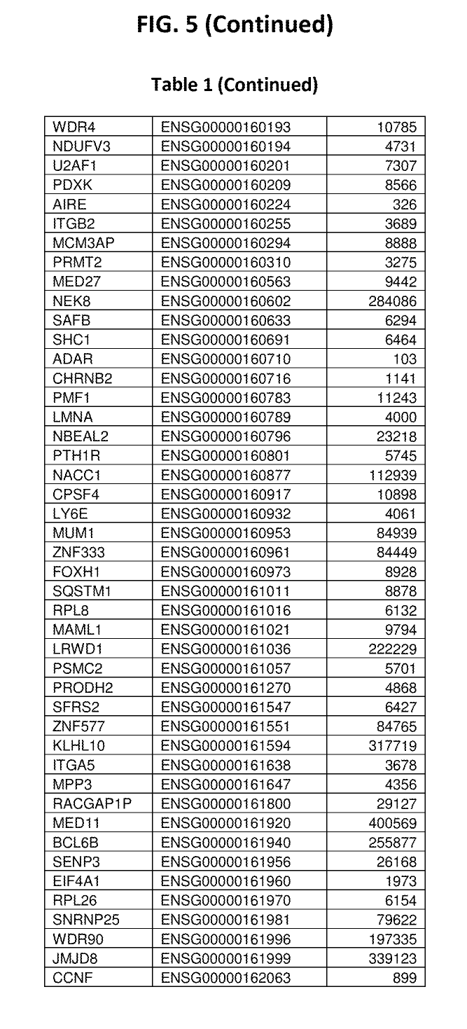

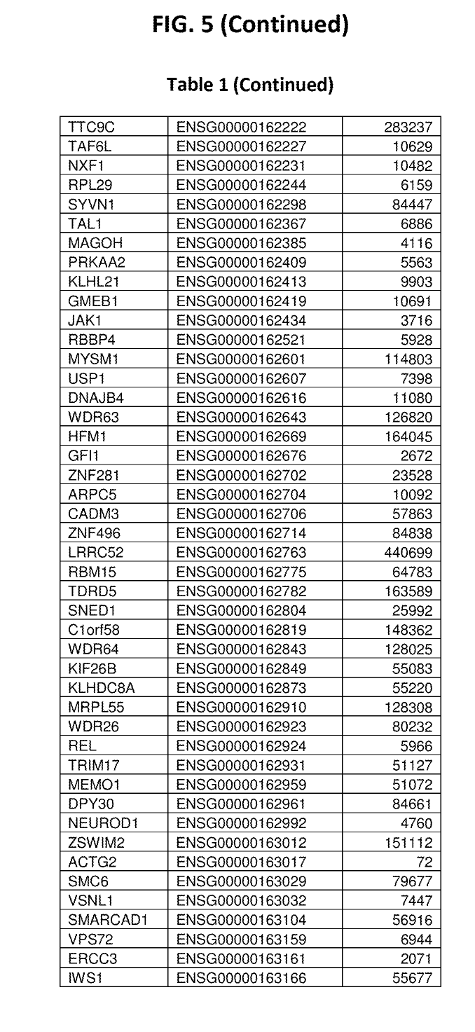

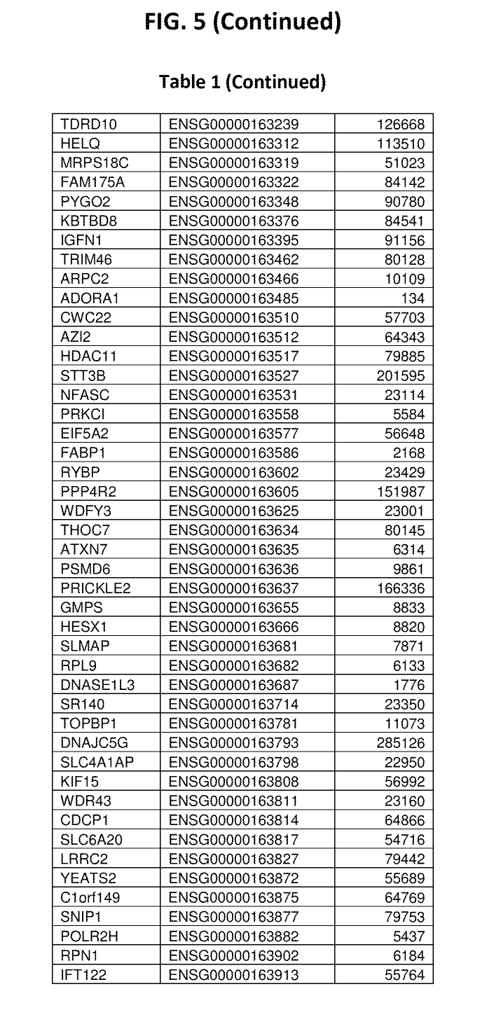

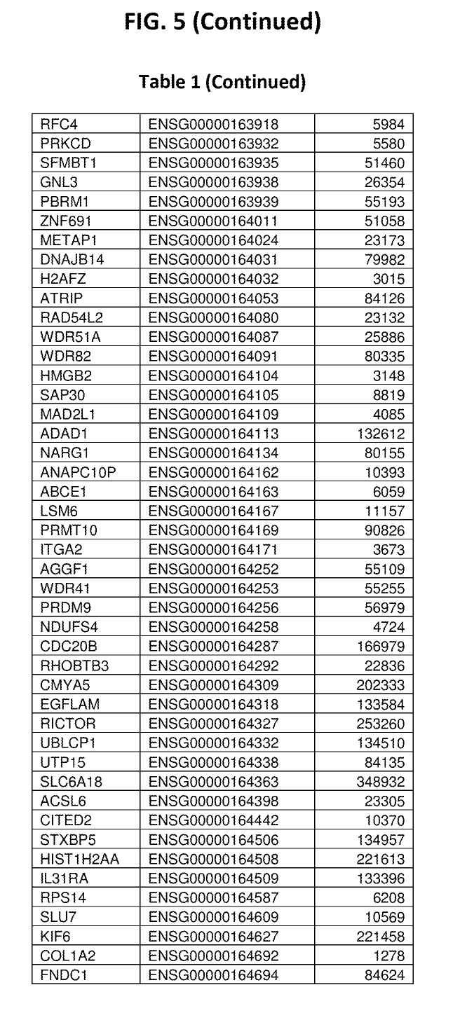

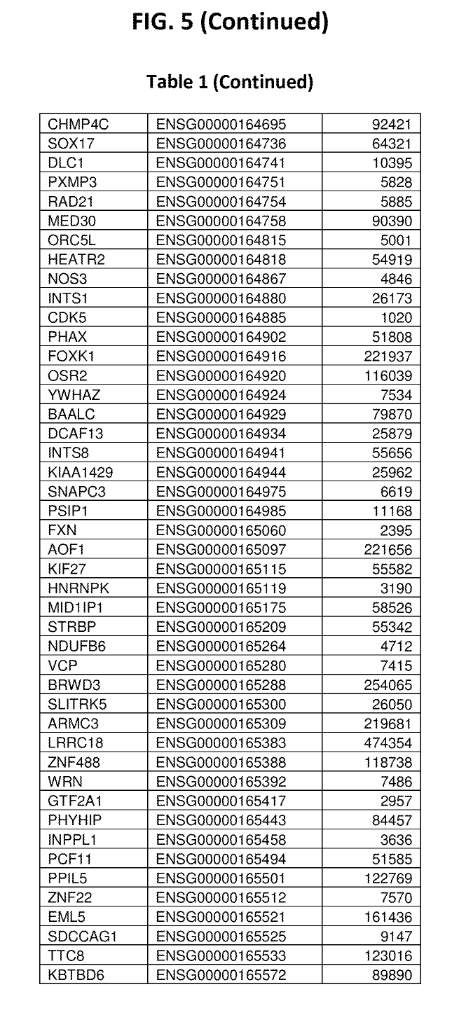

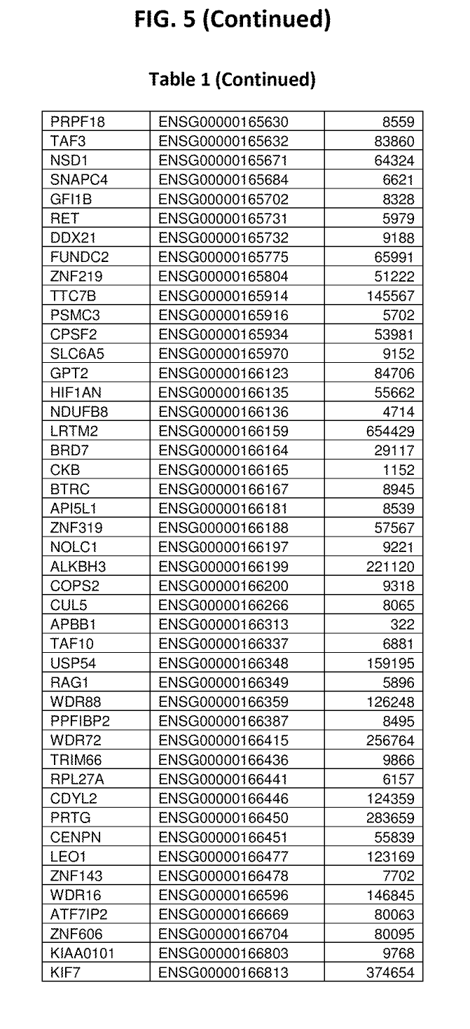

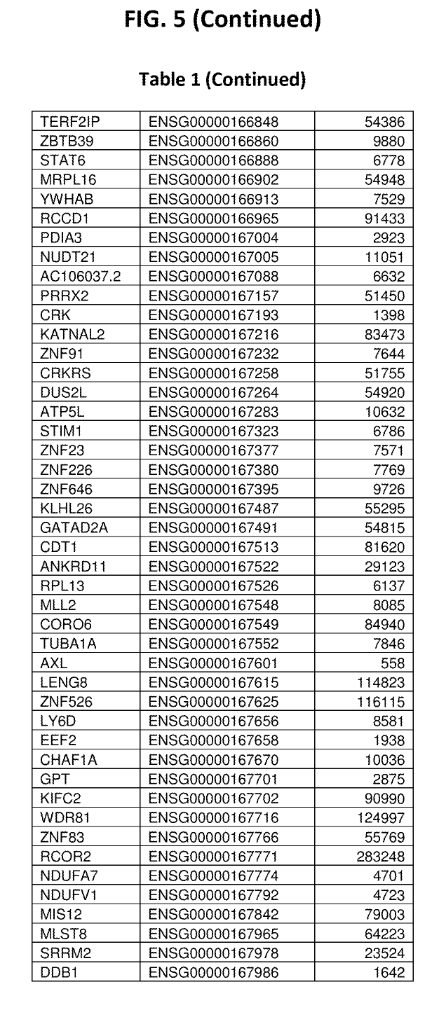

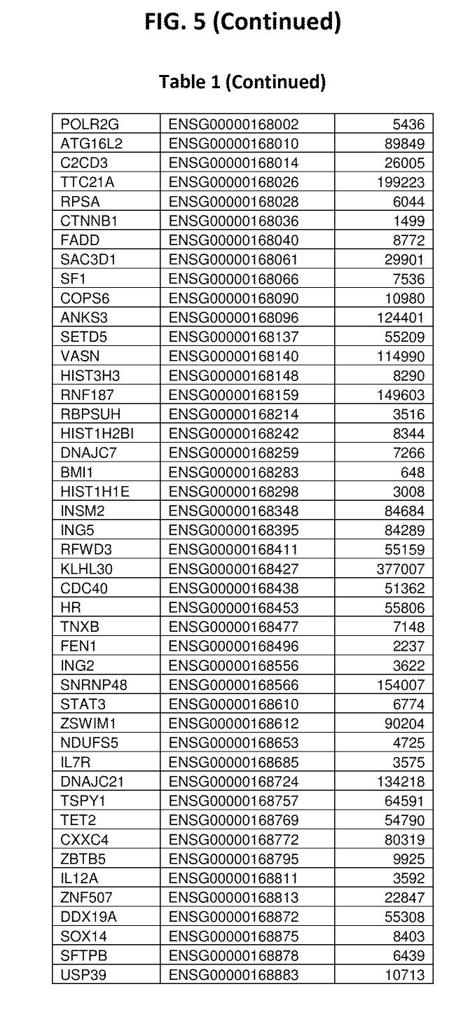

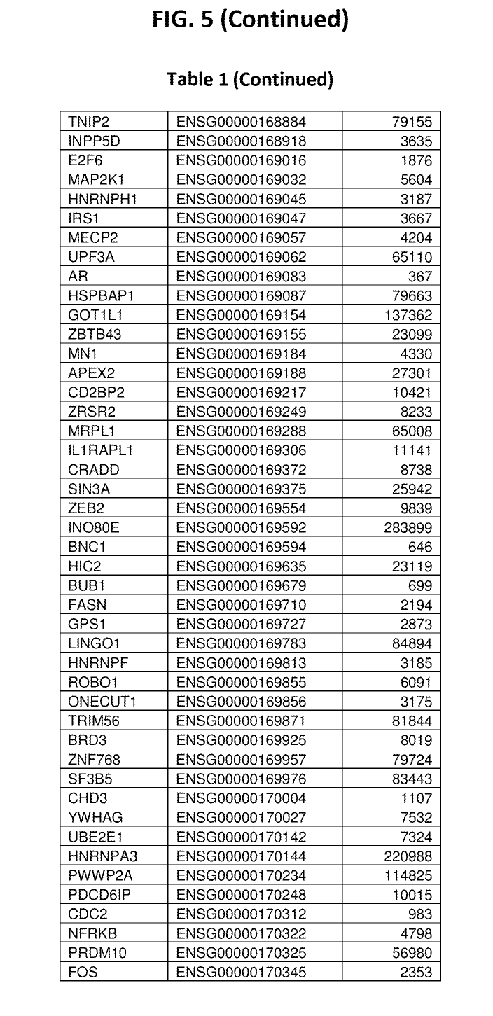

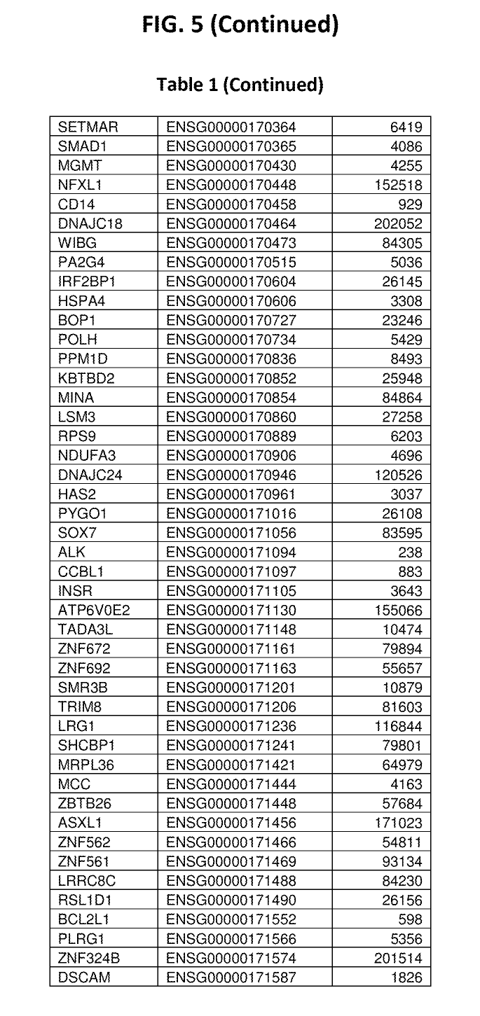

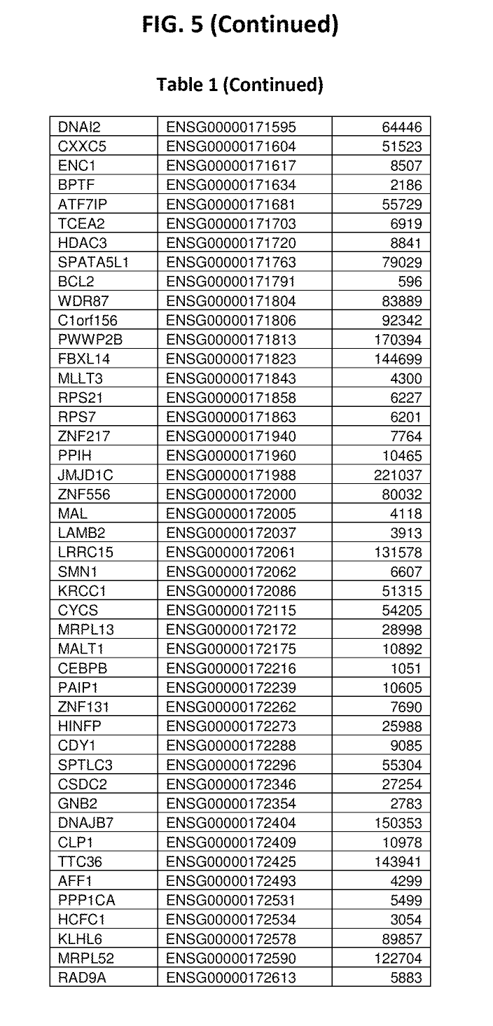

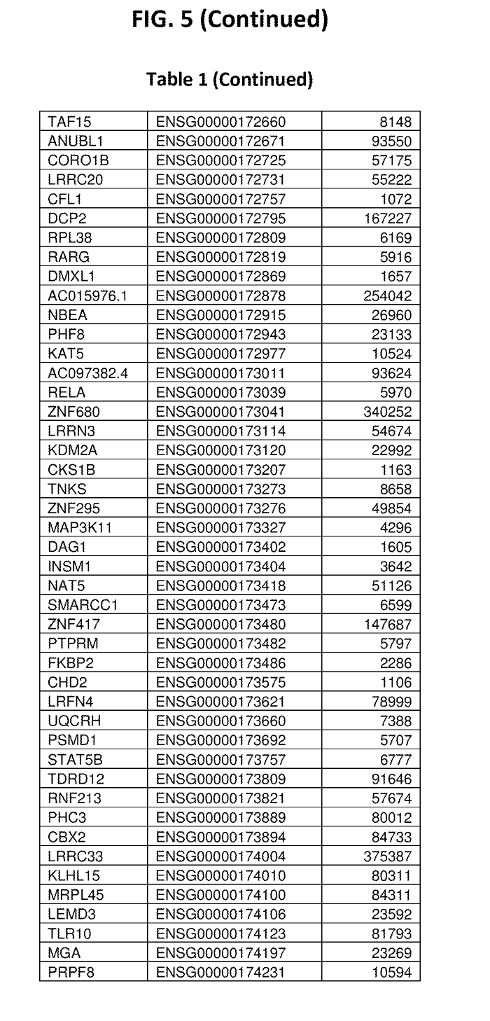

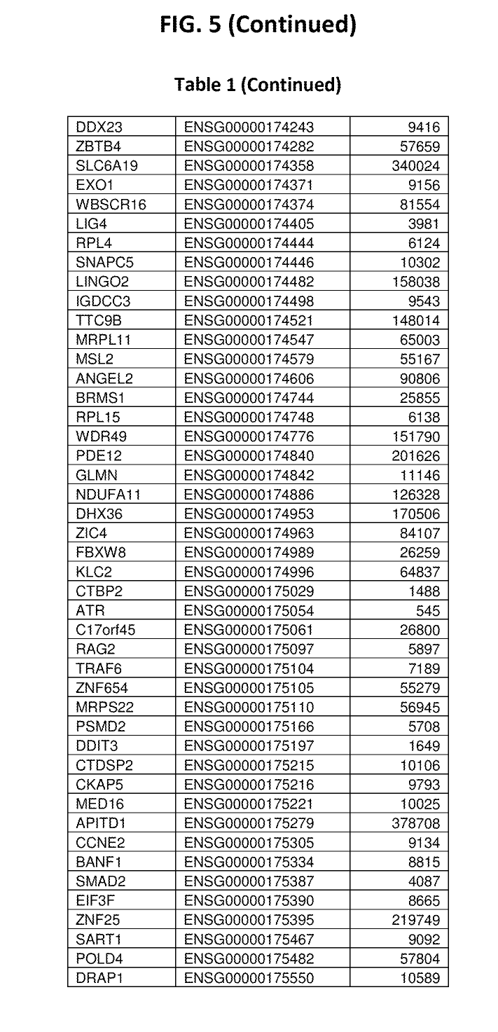

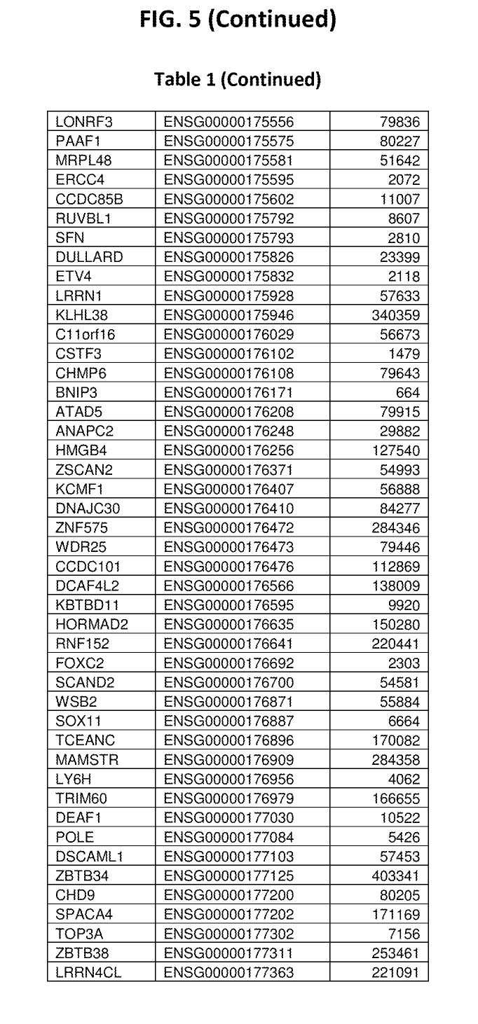

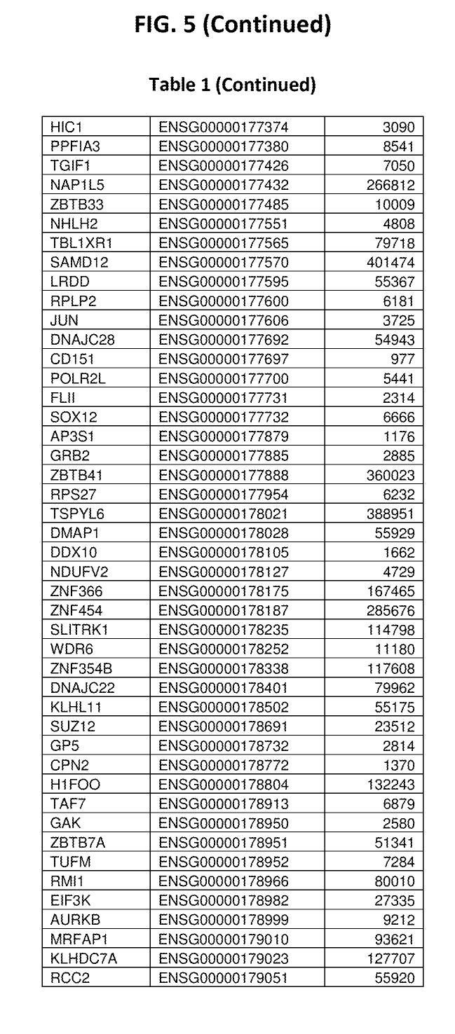

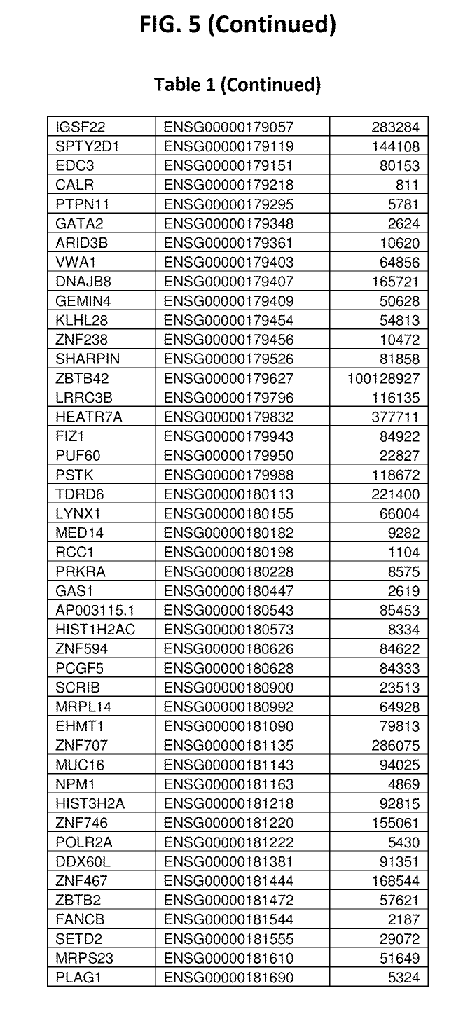

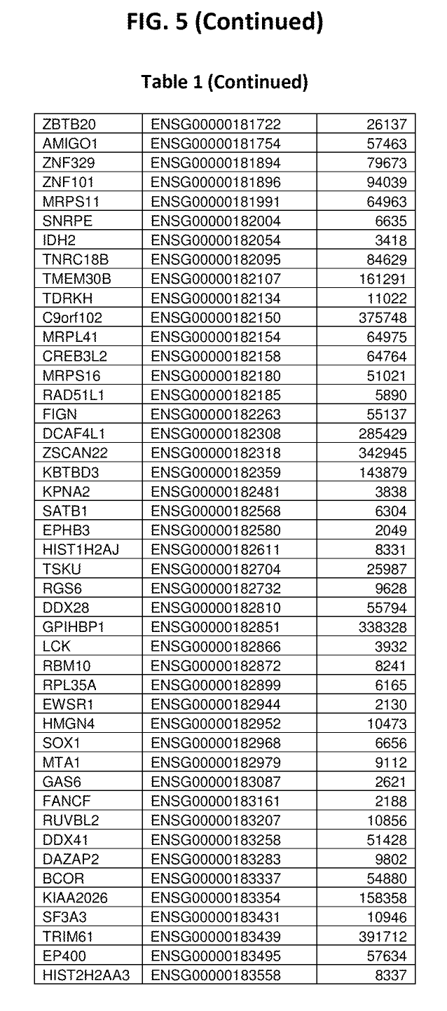

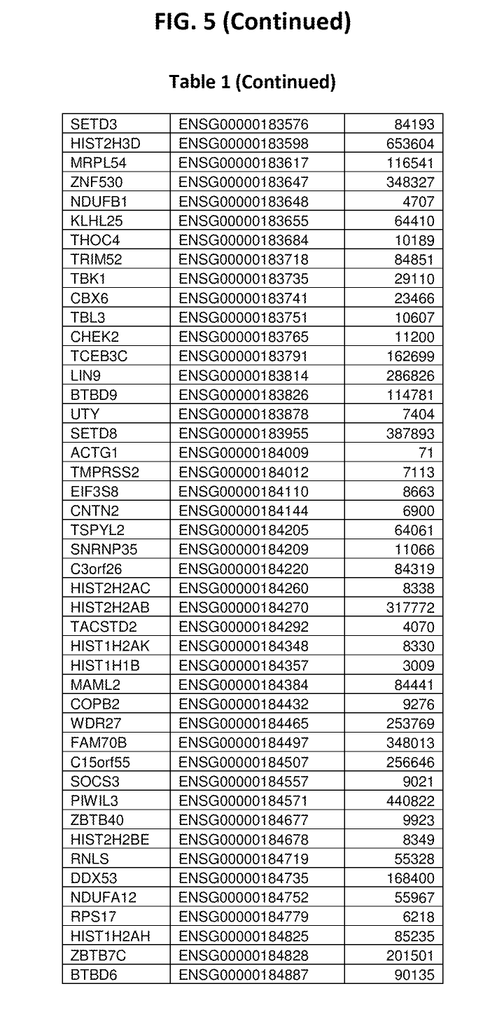

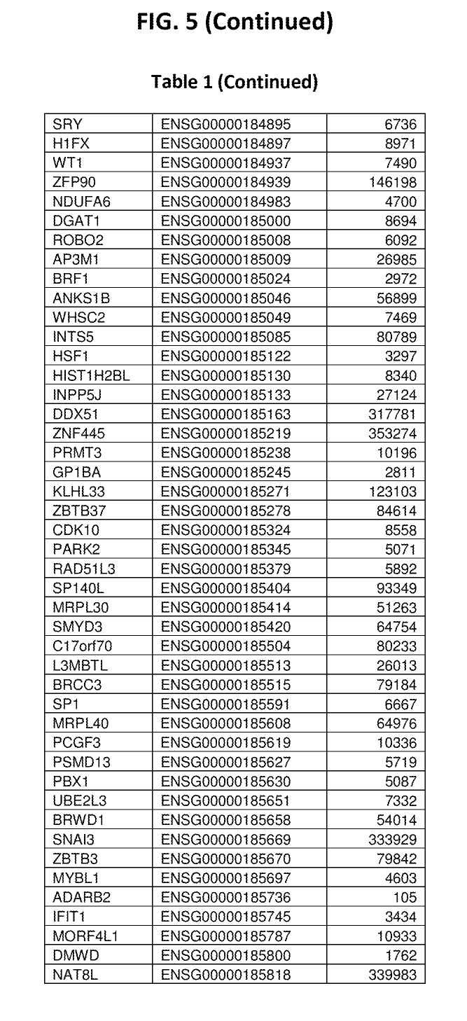

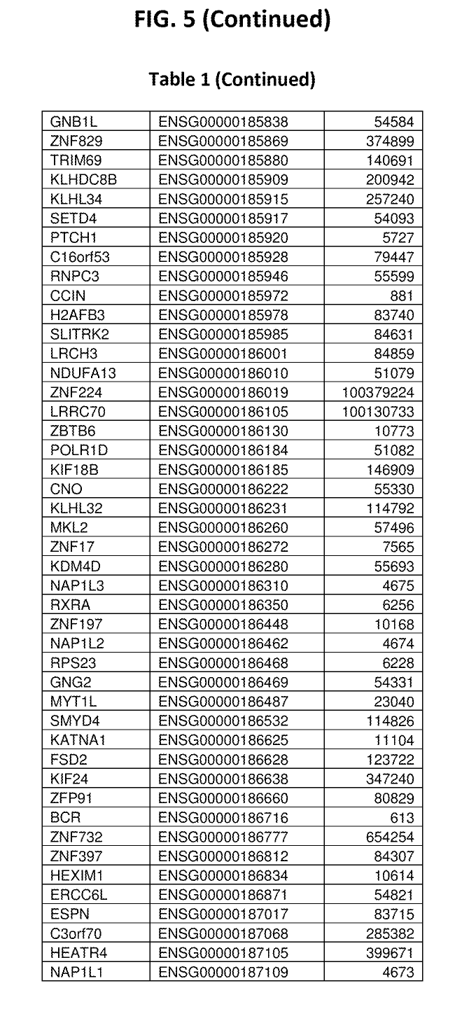

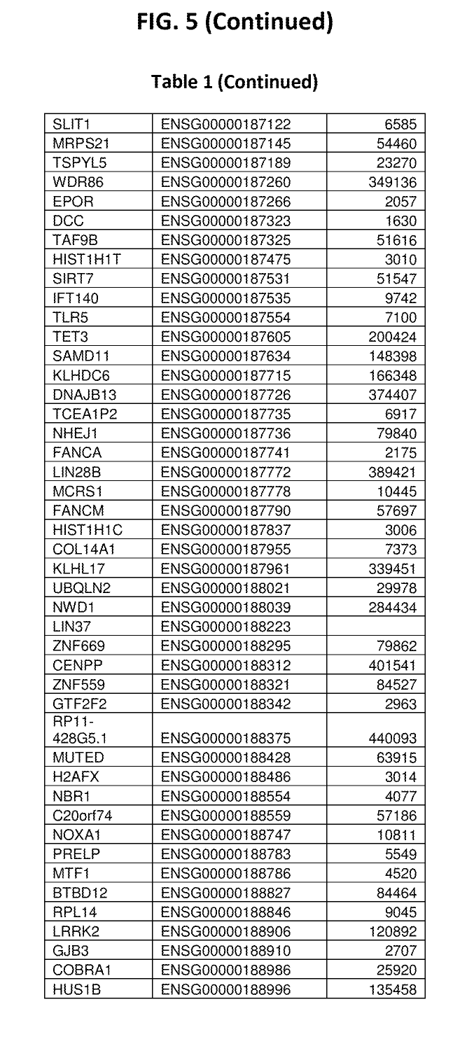

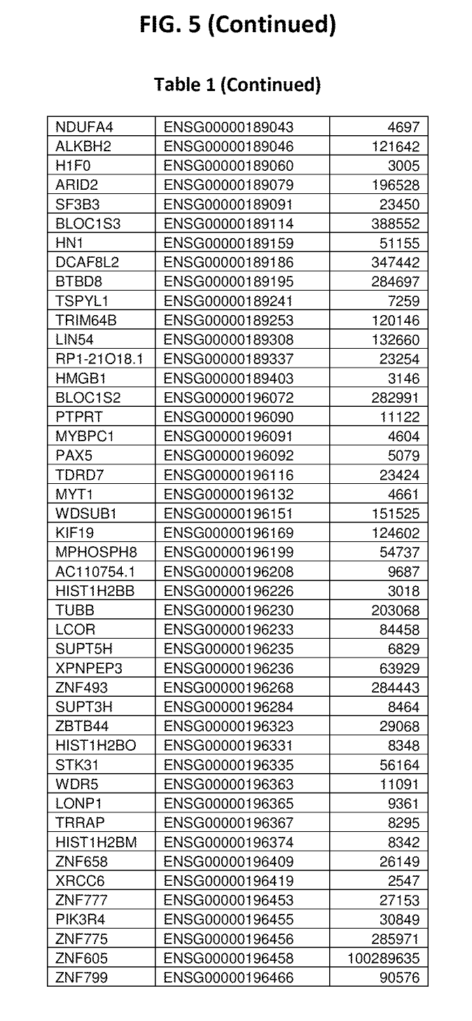

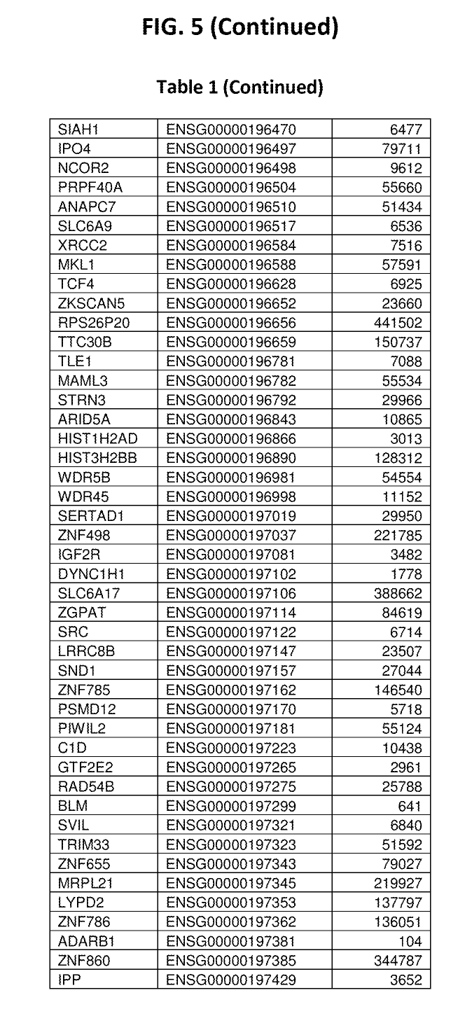

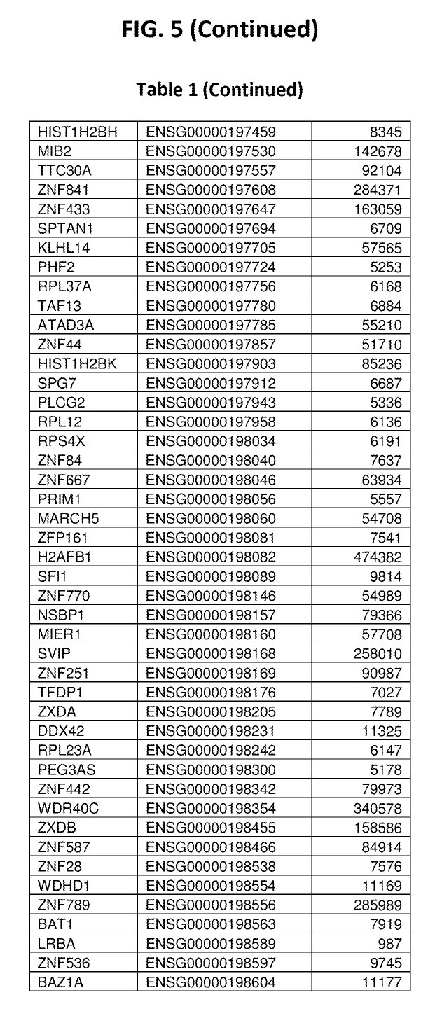

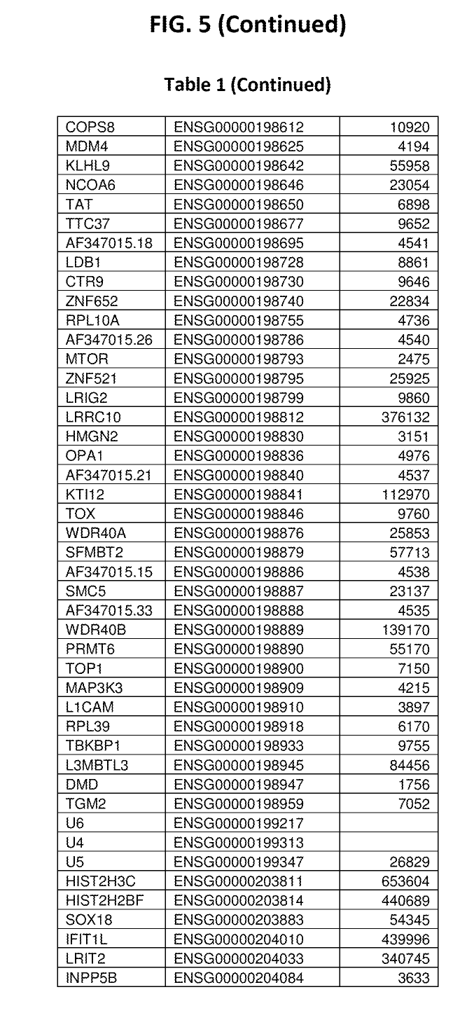

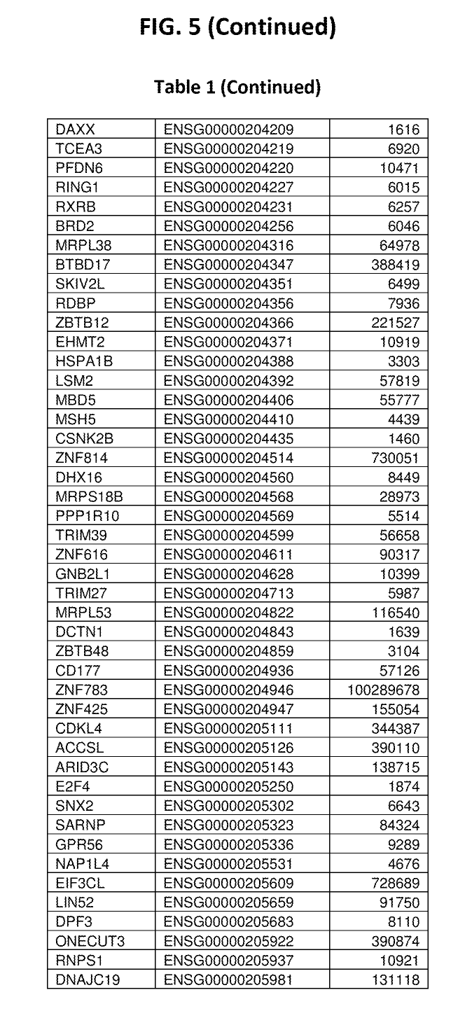

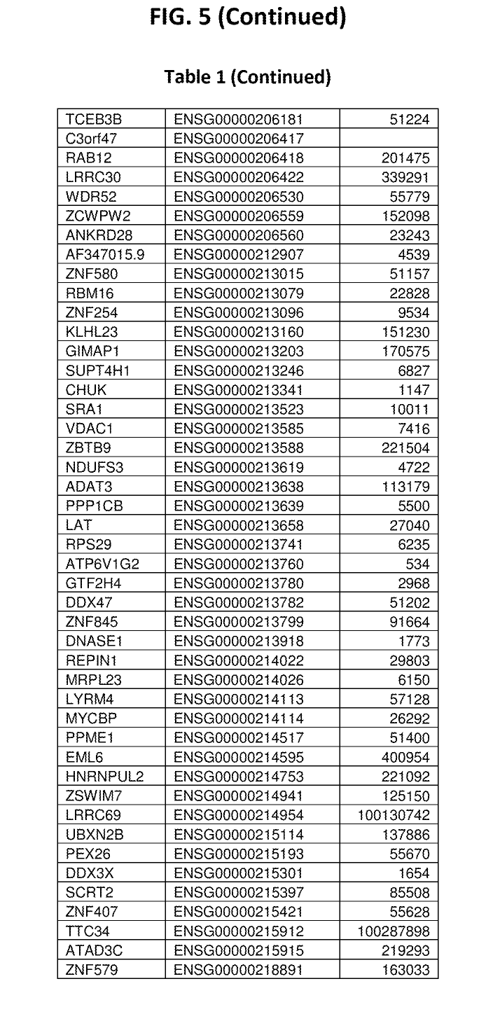

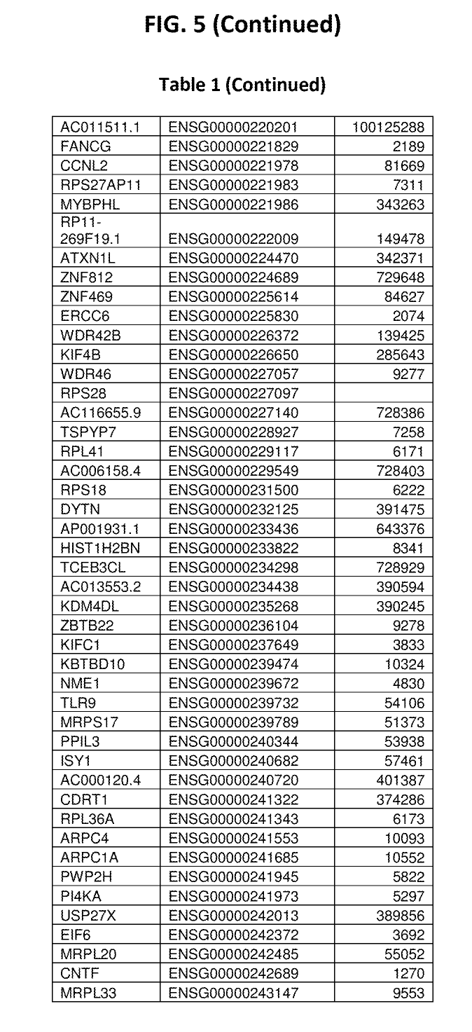

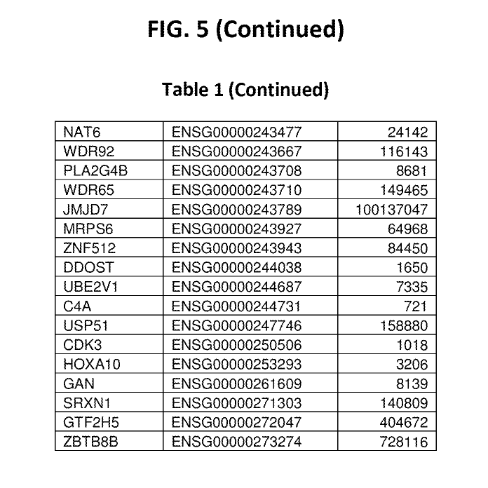

A process according to some embodiments is described with reference to FIG. 4, illustrating an exemplary process 400 of evaluating an enhancer component. In some embodiments, the process 400 may be embodied in a module, plugin, or component of the distributed system 200 shown in FIG. 2. In other embodiments, the process 400 may be embodied in a stand-alone application that performs the functions described herein.

Process 400 includes selecting an enhancer component associated with an enhancer at act 410. The enhancer component can be, for example, a protein or a nuclear protein. The enhancer can be, for example, a transcription factor a chromatin modulator. Exemplary enhancer components are those that bind to the chromatin domains provided in Table 1. In some embodiments, search queries can be executed on the distributed system 200 shown in FIG. 2, for example, of knowledge databases of proteins to select the enhancer component. According to one embodiment, the selection can be made from a pool of enhancer components. In some examples, a pool of enhancer components can be displayed to a user for selection in a user interface. In further examples, the user can select multiple enhancers component from the display. In some embodiment, the user interface can be configured to display multiple selections of enhancers to facilitate comparison of multiple enhancer components and/or potential treatment options that may be suitable.

In act 415, the selected enhancer component is evaluated. The evaluation can include acquiring knowledge of the relevance of the selected enhancer to a cell state. The cell state can include a state of health or disease or disorder and/or a state of differentiation. In some embodiments, search queries can be executed on the distributed system 200, for example, of knowledge databases to acquire the knowledge. One or more sources of information (e.g., external databases or document stores) may be used to search. Further, it should be appreciated that one or more knowledge-based systems may be used to analyze and extract knowledge from external databases or document stores for the purpose of evaluating SEs or any SE components.

In act 420, the selected enhancer component is classified based, at least in part, on the evaluation. The classification of the selected enhancer component can include classification as a candidate super-enhancer component. In some embodiments, the classification can also include classification as a candidate therapeutic or diagnostic target, such as the treatment or diagnosis of a subject having the cell state. In some embodiments, the genes can be classified on the distributed system 200, for example, by using metadata tags or some other appropriate designation system. Classification may be performed, for example, by any number or combination of machine learning systems, including but not limited to, artificial intelligence, neural networks, support vector machines, among others.

Attached to this provisional patent application is an appendix that describes various embodiments of the disclosure. These appendices form an integral part of the patent specification and describe various implementations according to aspects of various embodiments of the disclosure.

Appendix A describes in more detail methods and systems according to various aspects of the disclosure.

Any embodiment disclosed herein may be combined with any other embodiment, and references to “an embodiment,” “some embodiments,” “an alternate embodiment,” “various embodiments,” “one embodiment,” “at least one embodiment,” “this and other embodiments” or the like are not necessarily mutually exclusive and are intended to indicate that a particular feature, structure, or characteristic described in connection with the embodiment may be included in at least one embodiment. Such terms as used herein are not necessarily all referring to the same embodiment. Any embodiment may be combined with any other embodiment in any manner consistent with the aspects disclosed herein. References to “or” may be construed as inclusive so that any terms described using “or” may indicate any of a single, more than one, and all of the described terms. Furthermore, it will be appreciated that the systems and methods disclosed herein are not limited to any particular application or field, but will be applicable to any endeavor wherein a value is apportioned among several placements.

Where technical features in the drawings, detailed description, or any claim are followed by references signs, the reference signs have been included for the sole purpose of increasing the intelligibility of the drawings, detailed description, and claims. Accordingly, neither the reference signs nor their absence are intended to have any limiting effect on the scope of any claim placements.

Having now described some illustrative aspects of the disclosure, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the disclosure.

1. A method of treating a disorder in a subject, the method comprising administering to the subject an effective amount of a compound that decreases the activity, expression, or transcription of MED24, MED17, MED29, RBBP7, or RBBP4, thereby treating a disorder in the subject.

2. The method of claim 1, wherein the compound is a small molecule.

3. The method of claim 1, wherein the compound is a nucleic acid.

4. The method of claim 1, wherein the compound is an antibody.

5. The method of claim 1, wherein the disorder is cancer.

6. The method of claim 1, wherein the disorder is an immunological disorder.

7. A method of evaluating a gene and/or an associated super-enhancer comprising:
a) selecting a gene associated with a super-enhancer to provide a selected gene;
b) evaluating the selected gene for contribution to a cell state; and
c) responsive to the evaluation, classifying the selected gene and/or associated super-enhancer.

8. The method of claim 7, comprising classifying the selected gene as a candidate target gene, e.g., for the treatment or diagnosis of a subject having a disease.

9. The method of claim 7, wherein the selected gene is selected responsive to, or at least in part because of, its association with the super-enhancer.

10. The method of claim 7, further comprising providing a pool of genes associated with super-enhancers.

11. The method of claim 10, wherein said selected gene is selected from the pool.

12. The method of claim 7, wherein the cell state is selected from:
a state of health or disease or disorder; and
a state of differentiation.

13. The method of claim 7, wherein evaluating comprises acquiring knowledge of the selected gene relevant to the cell state.

14. The method of claim 7, wherein evaluating comprises acquiring knowledge of the association, correlation, or role of the selected gene to the cell state.

15. The method of claim 14, wherein the knowledge comprises knowledge of the effect of one or more of the following factors on the cell state or on a parameter associated with the cell state:
a) the effect of modulation,
b) the effect of gene targeting of the selected gene;
c) the effect or results of a synthetic lethal screen;
d) the effect of a genetic event involving the selected gene;
e) expression or hybrid capture sequencing data;
f) the effect of overexpressing a dominant negative mutant of the selected gene; or
g) the effect of reducing the copy number of the gene by genome editing or introducing an analog sensitive mutation into the gene by genome editing.

16. The method of claim 15, wherein the method comprises acquiring the knowledge from a database.

17. The method of claim 7, comprising determining if a super-enhancer is associated with the target gene in samples from one or a plurality of subjects.

18. The method of claim 7, further comprising confirming by performing experiments one of the effects listed in claim 9.

19. The method of claim 7, further comprising evaluating the effect of RNAi or a dominant negative mutation on the cell state.

20. A method of selecting a candidate super-enhancer component, comprising:
a) selecting an enhancer component that is associated with an enhancer to provide a selected enhancer component;
b) evaluating the selected enhancer component for contribution to a cell state;
c) responsive to the evaluation, classifying the selected enhancer component as a candidate super-enhancer component.

21. The method of claim 20, further comprising providing a pool of enhancer components.

22. The method of claim 21, wherein the selected enhancer component is selected from the pool.

23. The method of claim 20, wherein the method comprises the determining the identity of or finding an enhancer component by searching a database.

24. The method of claim 20, comprising classifying the super-enhancer component as a candidate therapeutic or diagnostic target.

25. The method of claim 20, wherein the cell state is selected from:
a state of health or disease or disorder; and
a state of differentiation.

26. The method of claim 20, wherein evaluating comprises acquiring knowledge of the selected enhancer component relevant to a cell state.

27. The method of claim 20, wherein evaluating comprises acquiring knowledge of the association, correlation, or role of the selected enhancer component to a cell state.

28. The method of claim 27, wherein the knowledge comprises knowledge of the effect of one or more of the following factors on the cell state or on a parameter associated with the cell state:
a) the effect of modulation of the level of gene expression or of a gene product of the gene encoding the selected enhancer component;
b) the effect of gene targeting of the gene encoding the selected enhancer component;
c) the effect of antagonizing the selected enhancer component; or
d) the effect of a genetic event involving the gene encoding the selected enhancer component.

29. The method of claim 28, wherein the method comprising acquiring the knowledge from a database.

30. The method of claim 20, further comprising evaluating whether the candidate super-enhancer component binds a super-enhancer.

31. The method of claim 20, further comprising evaluating whether down modulation of the candidate super-enhancer component disrupts super-enhancer function.

32. The method of claim 20, further comprising evaluating whether down modulation of the candidate super-enhancer component disrupts super-enhancer function more than it disrupts enhancer function.

33. The method of claim 20, comprising; determining if an candidate super-enhancer component is associated with a super-enhancer in samples from a plurality of subjects.

34. The method of claim 20, further comprising, confirming one of the effects listed in claim 28.

35. The method of claim 20, further comprising evaluating the effect of RNAi or a dominant negative mutation on the cell state.

36. A computer-implemented method of evaluating a gene and/or an associated super-enhancer comprising:
a) selecting a gene associated with a super-enhancer to provide a selected gene;
b) evaluating, on a computer, the selected gene for contribution to a cell state;
c) responsive to the evaluation, classifying the selected gene and/or associated super-enhancer.

37. A computer-implemented method of selecting a candidate super-enhancer component, comprising:
a) selecting an enhancer component that is associated with an enhancer to provide a selected enhancer component;
b) evaluating, on a computer, the selected enhancer component for contribution to a cell state;
c) responsive to the evaluation, classifying the selected enhancer component as a candidate super-enhancer component.

38. A system comprising:
a memory; and
a processing unit operative to:
a) select a gene associated with a super-enhancer to provide a selected gene;
b) evaluate the selected gene for contribution to a cell state; and
c) responsive to the evaluation, classify the selected gene and/or associated super-enhancer.

39. A system comprising:
a memory; and
a processing unit operative to:
a) select an enhancer component that is associated with an enhancer to provide a selected enhancer component;
b) evaluate, on a computer, the selected enhancer component for contribution to a cell state; and
c) responsive to the evaluation, classify the selected enhancer component as a candidate super-enhancer component.

40. A computer-readable medium comprising computer-executable instructions that, when executed on a processor of a computer, perform a method for evaluating a gene and/or an associate super-enhancer, comprising acts of:
a) selecting a gene associated with a super-enhancer to provide a selected gene;
b) evaluating the selected gene for contribution to a cell state;
c) responsive to the evaluation, classifying the selected gene and/or associated super-enhancer.

41. A computer-readable medium comprising computer-executable instructions that, when executed on a processor of a computer, perform a method for evaluating a candidate super-enhancer component, comprising acts of:
a) selecting an enhancer component;
b) evaluating the selected enhancer component for contribution to a cell state;
c) responsive to the evaluation, classifying the selected enhancer component as a candidate super-enhancer component.
